SEQUENCE LISTING (18 sequences)

SEQ ID NO: 1 - DNA, 24 bp, Artificial Sequence
  Description of Artificial Sequence Synthetic primer
tccaattcacactgaatgccagcc

SEQ ID NO: 2 - DNA, 24 bp, Artificial Sequence
  Description of Artificial Sequence Synthetic primer
aagcagtccttcttgtccaggtga

SEQ ID NO: 3 - DNA, 24 bp, Artificial Sequence
  Description of Artificial Sequence Synthetic primer
ctggccaatggcatggatctcaaa

SEQ ID NO: 4 - DNA, 24 bp, Artificial Sequence
  Description of Artificial Sequence Synthetic primer
atgaaatggcaaatcggctgacgg

SEQ ID NO: 5 - DNA, 24 bp, Artificial Sequence
  Description of Artificial Sequence Synthetic primer
acctgctagtgtgtgatgttccca

SEQ ID NO: 6 - DNA, 24 bp, Artificial Sequence
  Description of Artificial Sequence Synthetic primer
aggtggagagctttcagctcacat

SEQ ID NO: 7 - DNA, 24 bp, Artificial Sequence
  Description of Artificial Sequence Synthetic primer
tctgaataacgagaaggcgctggt

SEQ ID NO: 8 - DNA, 24 bp, Artificial Sequence
  Description of Artificial Sequence Synthetic primer
acaaggccactgactaggctgaaa

SEQ ID NO: 9 - DNA, 24 bp, Artificial Sequence
  Description of Artificial Sequence Synthetic primer
tccaattgagaccctggtggacat

SEQ ID NO: 10 - DNA, 24 bp, Artificial Sequence
  Description of Artificial Sequence Synthetic primer
tctcctatgtgctggctttggtga

SEQ ID NO: 11 - DNA, 24 bp, Artificial Sequence
  Description of Artificial Sequence Synthetic primer
agtggctaagggcatggagttctt

SEQ ID NO: 12 - DNA, 24 bp, Artificial Sequence
  Description of Artificial Sequence Synthetic primer
gggccaagccaaagtcacagattt

SEQ ID NO: 13 - DNA, 24 bp, Artificial Sequence
  Description of Artificial Sequence Synthetic primer
ttgctgatggcaacttcaactggg

SEQ ID NO: 14 - DNA, 24 bp, Artificial Sequence
  Description of Artificial Sequence Synthetic primer
tgtccagcccatgatggttctgat

SEQ ID NO: 15 - DNA, 24 bp, Artificial Sequence
  Description of Artificial Sequence Synthetic primer
ttgtggccttctttgagttcggtg

SEQ ID NO: 16 - DNA, 24 bp, Artificial Sequence
  Description of Artificial Sequence Synthetic primer
tcatccacagagcgatgttgtcca

SEQ ID NO: 17 - DNA, 24 bp, Artificial Sequence
  Description of Artificial Sequence Synthetic primer
tgatgctggtgctgagtatgtcgt

SEQ ID NO: 18 - DNA, 24 bp, Artificial Sequence
  Description of Artificial Sequence Synthetic primer
tctcgtggttcacacccatcacaa

(19) United States
(12) Patent Application Publication
(10) Pub. No.: US 2010/0266572 A1
(43) Pub. Date: Oct. 21, 2010

(54) TREATMENT AND PREVENTION OF ISCHEMIC INJURY USING ACTIVATED PROTEIN C

(76) Inventors: Robert Liwski, Halifax (CA); Michael Bezuhly, Halifax (CA)

Correspondence Address: FENWICK & WEST LLP, SILICON VALLEY CENTER, 801 CALIFORNIA STREET, MOUNTAIN VIEW, CA, 94041, US

(21) Appl. No.: 12/423,899

(22) Filed: Apr. 15, 2009

(51) Int. Cl. A61K 38/48; A61P 17/02

(52) U.S. Cl. 424/94.64

(57) ABSTRACT

Methods and compositions are provided for treating or preventing ischemic injury in a tissue flap in order to reduce the incidence of flap necrosis. Some compositions comprise one or more of an activated protein C (APC), a functional fragment of an APC, an APC mimetic compound, and a derivative of APC. Some methods comprise administering to a subject a therapeutically effective amount of an agent comprising one or more of an activated protein C (APC), a functional fragment of an APC, an APC mimetic compound, and a derivative of APC.

### FIELD

The present disclosure relates to the field of medicine and, more particularly, to materials and methods for improving the outcome of surgical procedures.

### BACKGROUND

Necrosis of tissue flaps remains a major complication in reconstructive surgery despite numerous methods to address the problem. Pharmacologic efforts to preserve the existing microcirculation have included the use of vasodilatory and anti-platelet agents, as well as antibodies against adhesion molecules and cytokines involved in leukocyte trafficking and microthrombus formation (Merchant et al., Am J Physiol Heart Circ Physiol; 284:H1260 (2003); Demirseren et al., J Reconstr Microsurg; 23:41 (2007); Pang et al., Ann Plast Surg; 22:293 (1989); Akan et al., Scand J Plast Reconstr Surg Hand Surg; 39:7 (2005); Engel et al., Ann Plast Surg; 58:456 (2007)). More recently, the administration of exogenous angiogenic factors has been shown to augment blood supply and improve flap survival (Kim et al., Plast Reconstr Surg; 120:1774 (2007); Carroll et al., Plast Reconstr Surg; 102:407 (1998); Padubidri et al., Ann Plast Surg; 37:604 (1996); Huang et al., Am J Physiol Heart Circ Physiol; 291:H127 (2006); Zheng et al., Plast Reconstr Surg; 121:59 (2008)). Among angiogenic agents examined, vascular endothelial growth factor (VEGF) has emerged as a key factor induced by ischemia (Ferrara et al., Nat Med; 9:669 (2003)).

The pathophysiology of ischemic injury is complex, involving multiple cell-cell interactions, signaling pathways and soluble factors (Jokuszies et al., J Reconstr Microsurg; 22:513 (2006)). Much of the initial injury observed in ischemia is produced by increased numbers of leukocytes trafficking into hypoxic tissues (Grace, P. A., Br J Surg; 81:637 (1994)). This inflammatory cell migration is mediated by the expression of pro-inflammatory mediators and surface adhesion molecules on the endothelium.

Surgical delay has traditionally been used to minimize inflammatory complications in tissue flaps. With this technique, attachment of a tissue flap is delayed for a period of days or weeks relative to an initial surgical procedure. Surgical delay has been shown to have early and late benefits that maintain the pre-existing microcirculation and promote angiogenesis respectively (Banbury et al., Plast Reconstr Surg; 104:730 (1999); Morris et al., Plast Reconstr Surg; 95:526 (1995); Kharbanda et al., Circulation; 103:1624 (2001); Yadav et al., Hepatology; 30:1223 (1999); Lefer et al., Cardiovasc Res; 32:743 (1996); Murphy et al., Br J Plast Surg; 38:272 (1985); Tepper et al., Blood; 105:1068 (2005); Park et al., Plast Reconstr Surg; 113:284 (2004)). Surgical delay, however, has the disadvantage of requiring an additional surgical procedure which may be associated with increased surgical morbidity and cost (Ercocen et al., Dermatol Surg; 29:692 (2003)).

Pharmacologic approaches that would avoid surgical morbidity in tissue flaps have met with limited success, due in part to their focus on single targets in the cascade of events leading to tissue damage. Accordingly, there is a need in the art for therapies that would decrease the incidence of tissue flap necrosis associated with reconstructive surgery.

Activated protein C (APC) is a serine protease having a molecular weight of about 56 kD. The inactive precursor, protein C, is a vitamin K-dependent glycoprotein synthesized by the liver and endothelium and is found in plasma. Activation of protein C occurs on the endothelial cell surface and is triggered by a complex formed between thrombin and thrombomodulin (Esmon et al., Thromb Haem; 78:70-74 (1997); Boffa et al., Lupus; 7:Suppl, 2-5 (1998)). APC is a potent natural anticoagulant found in serum that has been used in the treatment of severe sepsis (Bernard et al., N Engl J Med; 344:699 (2001); U.S. Pat. No. 4,775,624). APC possesses cytoprotective and anti-inflammatory activities. APC's cytoprotective properties are mediated by APC's engagement of its receptor, endothelial protein C receptor (EPCR) (Esmon C T, Curr Opin Hematol; 13:382 (2006); Vu et al., Cell; 64:1057 (1991)). By signaling through EPCR and protease activated receptor-1 (PAR-1), APC inhibits the transcriptional regulator NF-κB (Joyce et al., J Biol Chem; 276:11199 (2001); Franscini et al., Circulation; 110:2903 (2004)). The inhibition of NF-κB decreases the production of TNF-α required for upregulation of adhesion molecules such as intercellular adhesion molecule (ICAM)-1 (Barnes, P. J., Int J Biochem Cell Biol; 29:867 (1997)).

The present disclosure describes the first known use of APC for the reduction and prevention of ischemic injury in tissue flaps.

### SUMMARY

The present disclosure addresses long-felt needs in the field of medicine by providing compositions and methods for treating and preventing ischemic injury in tissue flaps.

Methods and compositions are provided herein for treating or preventing ischemic injury in a tissue flap in a subject, the methods comprising administering to the subject a therapeutically effective amount of an agent comprising one or more of: (i) an activated protein C (APC), (ii) a functional fragment of an APC, (iii) an APC mimetic compound, and (iv) a derivative of APC, optionally in admixture with a pharmaceutically acceptable carrier.

In certain aspects, a therapeutic composition is provided comprising one or more of: (i) an activated protein C (APC), (ii) a functional fragment of an APC, (iii) an APC mimetic compound, and (iv) a derivative of APC; optionally in admixture with a pharmaceutically acceptable carrier or additive.

In further aspects, a therapeutic composition is provided comprising one or more of: (i) an activated protein C (APC), (ii) a functional fragment of an APC, (iii) an APC mimetic compound, and (iv) a derivative of APC; optionally in admixture with a pharmaceutically acceptable carrier or additive, wherein the composition treats or prevents ischemic injury in a tissue flap in a subject.

In various aspects, the tissue flap is used for reconstructive surgery.

In various aspects, a first and a second surgical procedure are performed on a subject, the second surgical procedure comprising reconstructive surgery using a tissue flap and the first surgical procedure causing the subject to need reconstructive surgery, wherein reconstructive surgery using the tissue flap is delayed for a time interval of days to weeks following the first surgical procedure.

In further aspects, a first and a second surgical procedure are performed on a subject, the second surgical procedure comprising reconstructive surgery using a tissue flap and the first surgical procedure causing the subject to need reconstructive surgery, wherein reconstructive surgery using the tissue flap is performed contemporaneously with the first surgical procedure.

In various aspects, a first and a second surgical procedure are performed on a subject, the second surgical procedure comprising reconstructive surgery using a tissue flap and the first surgical procedure causing the subject to need reconstructive surgery, wherein the first surgical procedure is treating a wound or soft tissue defect resulting from cancer ablation.

In certain aspects, a kit is provided comprising one or more of: (i) an activated protein C (APC), (ii) a functional fragment of an APC, (iii) an APC mimetic compound, and (iv) a derivative of APC; optionally in admixture with a pharmaceutically acceptable carrier or additive.

In further aspects, a kit is provided comprising one or more of: (i) an activated protein C (APC), (ii) a functional fragment of an APC, (iii) an APC mimetic compound, and (iv) a derivative of APC; optionally in admixture with a pharmaceutically acceptable carrier or additive, wherein the kit is used to treat or prevent ischemic injury in a tissue flap in a subject.

### BRIEF DESCRIPTION OF THE DRAWINGS

FIG. 1. Schematic representation of the experimental designs for the postoperative (n=12 per APC or control group, panel A), late preoperative (n=5 per group, panel B), and early preoperative (n=5 per group, panel C) groups. Injection time-points are indicated by arrows.

FIG. 2. Representative postoperative day 7 appearance of ischemic skin flaps from postoperatively treated control (panel A) and APC group (panel B) rats. Clear demarcation of necrotic and viable regions is observed. Flap viability was significantly improved by APC treatment compared to PBS (panel C; ***p<0.001).

FIG. 3. Hematoxylin and eosin staining of skin flap dermis on postoperative day 2 (scale bar=50 μm). Untreated rat skin (panel A). Increased numbers of polymorphonuclear cells (arrows) are noted in control rat skin (panel B) compared to APC-treated rat skin (panel C). This difference in polymorphonuclear cells was significant (panel D, *p<0.05).

FIG. 4. Hematoxylin and eosin staining of skin flap panniculus carnosus on postoperative day 2 (scale bar=50 μm). Untreated rat panniculus carnosus (panel A). Note the loss of eosinophilia and increased fragmentation of striated muscle fibers with only a few isolated viable fibers (arrows) remaining in the control rat panniculus carnosus (panel B) compared to that of the APC-treated (panel C) rats. A significantly larger percentage of muscle fibers were found to be viable in the APC-treated skin flaps compared to control skin flaps (panel D, ***p<0.001).

FIG. 5. Periodic acid Schiff (PAS) staining (left column) and factor VIII-related antigen immunostaining (right column) of skin flaps on postoperative day 7 (scale bar=50 μm). A greater density of positively staining blood vessels are observed in APC-treated animals (panel C) compared to control (panel B) or untreated (panel A) animals. These differences in vessel numbers were significant (panel D, *p<0.05).

FIG. 6. Real-time PCR analysis of pro-inflammatory gene transcript levels. Downregulation of ICAM-1 and TNF-α transcript levels was noted at 3 hours and 24 hours, respectively, in APC-treated (black) compared to control (white) specimens (*p<0.05). Transcript levels were expressed relative to levels in untreated specimens (baseline=1).

FIG. 7. Real-time PCR analysis of pro-angiogenic and apoptotic gene transcript levels. A greater increase in Egr-1 and VEGFR2 transcript levels was noted at 3 hours and 24 hours, respectively, in APC-treated (black) compared to control (white) specimens (*p<0.05; **p<0.01). Similarly, an increase in Bcl-2 transcript levels above baseline was noted at 24 hours in APC-treated compared to control specimens (*p<0.05). Transcript levels were expressed relative to levels in untreated specimens (baseline=1).

FIG. 8. Representative postoperative day 7 appearance of ischemic skin flaps from preoperative experimental groups. Compared to control treatment (panel A), late preoperative APC treatment (panel B) led to flap hemorrhage, while early preoperative APC treatment (panel C) led to near-complete flap survival (panel D; ns=not significant; ***p<0.001).

### DETAILED DESCRIPTION

The descriptions of various aspects of the present disclosure are presented for purposes of illustration, and are not intended to be exhaustive or to limit the claimed methods to the forms disclosed. Persons skilled in the relevant art can appreciate that many modifications and variations are possible in light of the aspect teachings.

It should be noted that the language used herein has been principally selected for readability and instructional purposes, and it may not have been selected to delineate or circumscribe the inventive subject matter. Accordingly, the disclosure is intended to be illustrative, but not limiting, of the scope of claimed methods.

It must be noted that, as used in the specification, the singular forms “a,” “an” and “the” include plural referents unless the context clearly dictates otherwise.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

Methods and compositions are provided herein for treating or preventing ischemic injury in a tissue flap in a subject, the methods comprising administering to the subject a therapeutically effective amount of an agent comprising one or more of; (i) an activated protein C (APC), (ii) a functional fragment of an APC, (iii) an APC mimetic compound, and (iv) a derivative of APC, optionally in admixture with a pharmaceutically-acceptable carrier.

The term “tissue flap” means a vascularized section of living tissue that survives based on its own blood supply. A tissue flap may be a pedicled flap or a free flap. A tissue flap may contain one or more tissue types.

The term “pedicled flap” means a flap that contains the blood vessels that supply blood to the flap. These blood vessels are not severed from their original location in the body when the flap is transferred.

The term “free flap” means a flap in which the blood vessels that conduct blood into and away from the flap are cut so that the flap may be transferred to a target site. To reestablish blood flow to the flap, blood vessels at the target site are connected to the flap's vessels.

The term “skin flap” means a skin and subcutaneous tissue flap that survives based on its own blood supply. Skin flaps may be used for wound coverage when inadequate vascularity of the wound bed prevents skin grafting.

The term “fascia flap” means a sheet or band of fibrous connective tissue enveloping, separating, or binding together muscles, organs, and other soft structures of the body that survives based on its own blood supply.

The term “soft tissue defect” means any damage or defect occurring in the soft tissues (i.e., skin, muscle, fat, fibrous tissue, blood vessels, or other supporting tissue of the body) regardless of its cause.

The term “autologous”, with respect to transplantation, refers to a cell, tissue, organ, body part, etc. in which the donor and the recipient of the transplant are one and the same individual.

The term “heterologous”, with respect to transplantation, refers to a cell tissue, organ, body part, etc. in which the donor and the recipient of the transplant are different individuals.

The term “treating” refers to any indicia of success in the treatment or amelioration or prevention of the condition or disorder, including any objective or subjective parameter such as abatement; remission; or diminishing of symptoms; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present disclosure to prevent or delay, to alleviate, or to arrest or inhibit development of the symptoms associated with a condition or disorder as described herein. The term “therapeutic effect” refers to the reduction, elimination, or prevention of the condition or disorder, or side effects of the condition or disorder in the subject. “Treating” or “treatment” using the methods of the present disclosure includes preventing the onset of symptoms in a subject that can be at increased risk of a condition or disorder as described herein, but does not yet experience or exhibit symptoms, inhibiting the symptoms of a condition or disorder (slowing or arresting its development), providing relief from the symptoms or side effects of a condition or disorder (including palliative treatment), and relieving the symptoms of a condition or disorder (causing regression). Treatment can be prophylactic (to prevent or delay the onset of the condition or disorder, or to prevent the manifestation of clinical or subclinical symptoms thereof) or therapeutic suppression or alleviation of symptoms after the manifestation of the condition or disorder.

The term “ameliorating” refers to any therapeutically beneficial result in the treatment of a condition or disorder, e.g., ischemic injury in tissue flaps following reconstructive surgery, including prophylaxis, lessening in the severity or progression, remission, or cure thereof.

In general, the phrase “well tolerated” refers to the absence of adverse changes in health status that occur as a result of the treatment and would affect treatment decisions.

The term “sufficient amount” means an amount sufficient to produce a desired effect, e.g., an amount of time sufficient to reduce the incidence of hemorrhage during or after reconstructive surgery.

The term “therapeutically effective amount” is an amount that is effective to ameliorate a symptom of a condition or disorder. A therapeutically effective amount can be a “prophylactically effective amount” as prophylaxis can be considered therapy.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but can include other elements not expressly listed or inherent to such process or method. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

### Activated Protein C

Activated protein C (APC) is a potent natural anticoagulant found in serum that has been used in the treatment of severe sepsis (Bernard et al., N Engl J Med; 344:699 (2001); U.S. Pat. No. 4,775,624). Protein C is a member of the class of vitamin K-dependent serine protease coagulation factors. Protein C was originally identified for its anticoagulant and profibrinolytic activities. Protein C circulating in the blood is an inactive zymogen that requires proteolytic activation to regulate blood coagulation through a complex natural feedback mechanism. Human protein C is primarily made in the liver as a single polypeptide of 461 amino acids. This precursor molecule is then post-translationally modified by (i) cleavage of a 42 amino acid signal sequence, (ii) proteolytic removal from the one-chain zymogen of the lysine residue at position 155 and the arginine residue at position 156 to produce the two-chain form (i.e., light chain of 155 amino acid residues attached by disulfide linkage to the serine protease-containing heavy chain of 262 amino acid residues), (iii) carboxylation of the glutamic acid residues clustered in the first 42 amino acids of the light chain resulting in nine gamma-carboxyglutamic acid (GIa) residues, and (iv) glycosylation at four sites (one in the light chain and three in the heavy chain). The heavy chain contains the serine protease triad of Asp257, His2 1 and Ser360.

Similar to most other zymogens of extracellular proteases and the coagulation factors, protein C has a core structure of the chymotrypsin family, having insertions and an N-terminus extension that enable regulation of the zymogen and the enzyme. Of interest are two domains with amino acid sequences similar to epidermal growth factor (EGF). At least a portion of the nucleotide and amino acid sequences for protein C from human, monkey, mouse, rat, hamster, rabbit, dog, cat, goat, pig, horse, and cow are known, as well as mutations and polymorphisms of human protein C (see GenBank accession P04070). Other variants of human protein C are known which affect different biological activities.

APC also possesses cytoprotective activities. These properties are mediated by APC engagement of its receptor, endothelial protein C receptor (EPCR) (Esmon CT, Curr Opin Hematol; 13:382 (2006); Vu et al., Cell; 64:1057 (1991)). By signaling through EPCR and protease activated receptor-1 (PAR-1), APC inhibits the transcriptional regulator NF-κB (Joyce et al., J Biol Chem; 276:11199 (2001); Franscini et al., Circulation; 110:2903 (2004)). The inhibition of NF-κB decreases the production of TNF-α required for upregulation of adhesion molecules such as intercellular adhesion molecule (ICAM)-1 (Barnes, P. J., Int J Biochem Cell Biol; 29:867 (1997)).

APC's ability to downregulate adhesion molecule and cytokine expression has been clearly shown to decrease leukocyte rolling and firm adherence. Several intravital microscopy studies have demonstrated that APC dramatically inhibits leukocyte trafficking (Bartolome et al., Shock; 29:384 (2008)). Decreased cytokine release and vascular adhesion molecule expression by endothelial cells is only partly responsible for the ability of APC to decrease leukocyte trafficking and tissue damage. APC has also been shown to protect endothelial barrier function, further preventing leukocyte infiltration and edema. APC maintains endothelial barrier integrity by inducing cytoskeleton rearrangements within the endothelial cell (Bae et al., Blood; 110:3909 (2007)). In addition, APC has been shown to downregulate the pro-apoptotic mediators Bax and caspase-3, and to upregulate anti-apoptotic Bcl-2 (Cheng et al., Nat Med; 9:338 (2003); Mosnier et al., Biochem J; 373:65 (2003)). Limited evidence suggests that APC may also be pro-angiogenic; however, the mechanism by which APC stimulates angiogenesis remains unclear (Xue et al., Clin Hemorheol Microcirc; 34:153 (2006); Jackson et al., Wound Repair Regen; 13:284 (2005)).

APC's unique combination of activities, as described above, make it beneficial in the prevention and reduction of flap ischemic injury.

Methods for making and isolating activated protein C are described in U.S. Pat. No. 4,775,624. Those methods are incorporated herein by reference.

Methods and compositions are provided herein for treating or preventing ischemic injury in a tissue flap in a subject, the methods comprising administering to the subject a therapeutically effective amount of an agent comprising one or more of; (i) an activated protein C (APC), (ii) a functional fragment of an APC, (iii) an APC mimetic compound, and (iv) a derivative of APC, optionally in admixture with a pharmaceutically-acceptable carrier.

In some aspects, methods and compositions are provided herein for treating or preventing ischemic injury in a tissue flap in a subject, the method comprising administering to the subject a therapeutically effective amount of APC.

In some aspects, methods and compositions are provided herein for treating or preventing ischemic injury in a tissue flap in a subject, the method comprising administering to the subject a therapeutically effective amount of human APC.

In further aspects, methods and compositions are provided herein for treating or preventing ischemic injury in a tissue flap in a subject, the method comprising administering to the subject a therapeutically effective amount of a functional fragment of APC.

In further aspects, methods and compositions are provided herein for treating or preventing ischemic injury in a tissue flap in a subject, the method comprising administering to the subject a therapeutically effective amount of an APC mimetic compound.

In further aspects, methods and compositions are provided herein for treating or preventing ischemic injury in a tissue flap in a subject, the method comprising administering to the subject a therapeutically effective amount of an APC derivative.

APC functional fragments may also be used according to the present disclosure. Suitable functional fragments of an APC may be produced by cleaving purified natural APC or recombinant APC with well known proteases such as trypsin and the like, or more preferably, by recombinant DNA techniques or peptide/polypeptide synthesis. Such functional fragments may be identified by generating candidate fragments and assessing biological activity by, for example, assaying for activation of MMP-2, promotion of repair of a wounded endothelial monolayer and/or angiogenesis in chicken embryo chorio-allantoic membrane (CAM), or in a manner similar to that described in the examples provided herein. Preferably, functional fragments will be of 5 to 100 amino acids in length, more preferably, of 10 to 30 amino acids in length. The functional fragments may be linear or circularized and may include modifications of the amino add sequence of the native APC sequence from whence they are derived (e.g., amino acid substitutions, deletions, and additions of heterologous amino acid sequences). The functional fragments may also be glycosylated by methods well known in the art and which may comprise enzymatic and non-enzymatic means.

APC mimetic compounds may also be used according to the present disclosure. Suitable APC mimetic compounds (i.e., compounds which mimic the function of APC) may be designed using any of the methods well known in the art for designing mimetics of peptides based upon peptide sequences in the absence of secondary and tertiary structural information (Kirshenbaun et al., Curr Opin Struct Biol; 9:530-535 (1999)). For example, peptide mimetic compounds may be produced by modifying amino acid side chains to increase the hydrophobicity of defined regions of the peptide (e.g., substituting hydrogens with methyl groups on aromatic residues of the peptides), substituting amino acid side chains with non-amino acid side chains (e.g., substituting aromatic residues of the peptides with other aryl groups), and substituting amino- and/or carboxy-termini with various substituents (e.g., substituting aliphatic groups to increase hydrophobicity). Alternatively, the mimetic compounds may be so-called peptoids (i.e., non-peptides) which include modification of the peptide backbone (i.e., by introducing amide bond surrogates by, for example, replacing the nitrogen atoms in the backbone with carbon atoms), or include N-substituted glycine residues, one or more D-amino acids (in place of L-amino acid(s)) and/or one or more α-amino acids (in place of β-amino acids or γ-amino acids). Further mimetic compound alternatives include “retro-inverso peptides” where the peptide bonds are reversed and D-amino acids assembled in reverse order to the order of the L-amino acids in the peptide sequence upon which they are based, and other non-peptide frameworks such as steroids, saccharides, benzazepine1,3,4-trisubstituted pyrrolidinone, pyridones and pyridopyrazines. Suitable mimetic compounds may also be designed/identified by structural modeling/determination, by screening of natural products, the production of phage display libraries (Sidhu et al., Methods Enzymol; 328:333-363 (2000)), minimized proteins (Cunningham et al., Curr Opin Struct Biol; 7:457-462 (1997)), SELEX (aptamer) selection (Drolet et al., Comb Chem High Throughout Screen; 2:271-278 (1999)), combinatorial libraries and focused combinatorial libraries, virtual screening/database searching (Bissantz et al., J Med Chem; 43:4759-4767 (2000)), and rational drug design techniques well known in the art (Houghten et al., Drug Discovery Today; 5:276-285 (2000)).

APC derivatives may also be used according to the present disclosure. Suitable APC derivatives include peptides in which one or several amino acids have been derivatized by a chemical reaction. Examples of peptide derivatives according to the present disclosure are in particular those molecules in which the backbone or/and reactive amino acid side groups, e.g., free amino groups, free carboxyl groups or/and free hydroxyl groups have been derivatized. Specific examples of derivatives of amino groups are sulfonic acid or carboxylic acid amides, thiourethane derivatives and ammonium salts e.g. hydrochlorides. Examples of carboxyl group derivatives are salts, esters and amides. Examples for hydroxyl group derivatives are O-acyl or O-alkyl derivatives. Furthermore the term peptide derivative according to the present disclosure also includes those peptides in which one or several amino acids are replaced by naturally occurring or non-naturally occurring amino acid homologues of the 20 “standard” amino acids. Examples of such homologues are 4-hydroxyproline, 5-hydroxylysine, 3-methylhistidine, homoserine, ornithine, .beta.-alanine and 4-aminobutyric acid.

APC variants (mutants) may also be used according to the present disclosure. APC variants, as described in U.S. Pat. App. Pub. No. 2007/0042961, are incorporated herein by reference. APC normally has anticoagulant, anti-thrombotic, anti-inflammatory, and anti-apoptotic activities. In some instances, however, recombinant activated protein C mutants have markedly reduced anticoagulant activity, but retain near normal anti-apoptotic (cytoprotective) activity, so that the ratio of anti-apoptotic to anticoagulant activity is greater in the variants than it is in wild-type or endogenous activated protein C. APC variants are useful as inhibitors of apoptosis or cell death and/or as cell survival factors. In the case of variants having reduced anticoagulant activity, the risk of bleeding during treatment with APC is diminished.

### Reconstructive Surgery

Reconstructive surgery is generally performed on abnormal structures of the body, caused by birth defects, developmental abnormalities, trauma or injury, infection, tumors, or disease. It is generally performed to improve function, but may also be done to approximate a normal appearance. Cosmetic surgery is performed to reshape normal structures of the body to improve the patient's appearance and self-esteem.

Complications resulting from reconstructive and cosmetic surgery may include ischemic injury; infection; significant bruising and wound-healing difficulties; pain; edema; and problems related to anesthesia and surgery. The methods and compositions described herein improve the success of reconstructive surgery using tissue flaps by treating and preventing ischemic injury in the tissue flap.

Many common reconstructive and cosmetic surgery procedures result in painful swelling and bleeding where tissue flaps are used. In breast augmentation, breast reduction, mastopexy and gynecomastia procedures, for example, fluid accumulation and swelling may result possibly requiring subsequent corrective surgical procedures. In such procedures, skin of and around the nipple is separated and/or removed from the underlying breast tissue. A tissue flap is frequently necessary to compensate for the change in breast size and/or to gain access to underlying tissues for implantation or reduction.

Surgical delay has traditionally been used to minimize inflammatory complications in tissue flaps. According to the present disclosure, surgical delay may optionally be used in combination with APC administration. With this technique, attachment of a tissue flap is delayed for a period of days or weeks relative to an initial surgical procedure. Surgical delay has been shown to have early and late benefits that maintain the pre-existing microcirculation and promote angiogenesis respectively (Banbury et al., Plast Reconstr Surg; 104:730 (1999); Morris et al., Plast Reconstr Surg; 95:526 (1995); Kharbanda et al., Circulation; 103:1624 (2001); Yadav et al., Hepatology; 30:1223 (1999); Lefer et al., Cardiovasc Res; 32:743 (1996); Murphy et al., Br J Plast Surg; 38:272 (1985); Tepper et al., Blood; 105:1068 (2005); Park et al., Plast Reconstr Surg; 113:284 (2004)).

In various aspects, a tissue flap is used for reconstructive surgery.

In various aspects, a tissue flap is attached using microvascular surgical techniques.

In certain aspects, reconstructive surgery is used to treat a wound or soft tissue defect resulting from cancer ablation.

In some aspects, reconstructive surgery is used to treat a wound or soft tissue defect resulting from mastectomy, skin cancer excision, or head and neck cancer.

In some aspects, reconstructive surgery is used to treat traumatic injury.

In various aspects, a first and a second surgical procedure are performed on a subject, the second surgical procedure comprising reconstructive surgery using a tissue flap and the first surgical procedure causing the subject to need reconstructive surgery.

In further aspects, a first and a second surgical procedure are performed on a subject, the second surgical procedure comprising reconstructive surgery using a tissue flap and the first surgical procedure causing the subject to need reconstructive surgery, wherein reconstructive surgery using the tissue flap is delayed for a time interval ranging from days to weeks following the first surgical procedure.

In further aspects, a first and a second surgical procedure are performed on a subject, the second surgical procedure comprising reconstructive surgery using a tissue flap and the first surgical procedure causing the subject to need reconstructive surgery, wherein reconstructive surgery using the tissue flap is delayed for a time interval ranging from about 7 to about 21 days following the first surgical procedure.

In further aspects, a first and a second surgical procedure are performed on a subject, the second surgical procedure comprising reconstructive surgery using a tissue flap and the first surgical procedure causing the subject to need reconstructive surgery, wherein reconstructive surgery using the tissue flap is delayed for a time interval of about 7 days following the first surgical procedure.

In further aspects, a first and a second surgical procedure are performed on a subject, the second surgical procedure comprising reconstructive surgery using a tissue flap and the first surgical procedure causing the subject to need reconstructive surgery, wherein reconstructive surgery using the tissue flap is performed contemporaneously with the first surgical procedure.

In some aspects, a first and a second surgical procedure are performed on a subject, the second surgical procedure comprising reconstructive surgery using a tissue flap and the first surgical procedure causing the subject to need reconstructive surgery, wherein the first surgical procedure is treating a wound or soft tissue defect resulting from cancer ablation.

In certain aspects, a first and a second surgical procedure are performed on a subject, the second surgical procedure comprising reconstructive surgery using a tissue flap and the first surgical procedure causing the subject to need reconstructive surgery, wherein the wound or soft tissue defect results from mastectomy, skin cancer excision, or head and neck cancer.

In some aspects, a first and a second surgical procedure are performed on a subject, the second surgical procedure comprising reconstructive surgery using a tissue flap and the first surgical procedure causing the subject to need reconstructive surgery, wherein the first surgical procedure is treating traumatic injury.

According to the present disclosure, the administration of APC has therapeutic effect with various types of reconstructive surgery. Cosmetic surgery procedures such as rhytidectomy, browlift, otoplasty, blepharoplasty, rhinoplasty, facial implant, and hair replacement therapy will also benefit from the present disclosure. In such procedures, skin is lifted and underlying tissue and muscles are removed or manipulated. A tissue flap is frequently necessary to compensate for skin tissue loss and/or to gain access to the tissues and muscles beneath the skin.

In an abdominoplasty procedure, the abdomen is flattened by removing excess fat and skin and tightening muscles of the abdominal wall. Bleeding under the tissue flap and poor healing resulting in skin loss and scarring may occur, possibly requiring a second operation.

Reconstructive surgery procedures such as those to repair a birthmark, cleft palate, cleft lip, syndactyly, urogenital and anorectal malformations, craniofacial birth defects, ear and nasal deformities or vaginal agenesis similarly involve incisions and manipulations in skin and underlying tissues for the restoration of body features. A tissue flap is frequently necessary to compensate for skin tissue loss and/or to gain access to the tissues and muscles beneath the skin.

Similarly, reconstructive surgery to correct defects resulting from an injury such as a burn, infection, or disease such as skin cancer will also benefit from the compositions and methods of the present disclosure. For example, an oseomyocutaneous flap (a flap containing bone and soft tissue) is often used to reconstruct the skin following skin cancer excision. Thus, the present disclosure may be employed to reduce the swelling and scarring complications associated with such a procedure.

### Tissue Flaps

A flap is a section of living tissue that carries its own blood supply and is moved from one area of the body to another. Flap surgery can restore form and function to areas of the body that have lost skin, fat, muscle movement, and/or skeletal support.

Within the surgical literature, it has been known for at least thirty years that humans (as well as other mammals) possess self-contained expendable microvascular beds (Armstrong et al., Clin Plast Surg; 28:671-86 (2001); Buncke et al., Plast Reconstr Surg; 98:1122-3 (1996)). Examples in humans include the omentum, the temporoparietal fascia, and the transverse rectus abdominis myocutaneous tissue, among hundreds of others (Liebermann et al., Neth J Surg; 43:136-44 (1991); Brent et al., Plast Reconstr Surg; 76:177-88 (1985); Lorenzetti et al., J Reconstr Microsurg; 17:163-7 (2001); Serletti et al., Semin Surg Oncol; 19:264-71 (2000); Chang et al., Semin Surg Oncol; 19:211-7 (2000); Chen et al., Hand Clin; 15:541-54 (1999)). These microvascular beds are considered expendable because they can be removed with no residual disability. Similar expendable vascular beds occur in animal models (Hoyt et al., Lab Anim (NY); 30:26-35 (2001); Zhang et al., J Reconstr Microsurg; 17:211-21 (2001); Taylor et al., Plast Reconstr Surg; 89:181-215 (1992)). These microvascular beds are frequently composite tissues, such as bone and skin, muscle and skin, etc.

Microvascular beds may be removed, transferred to another location in the donor (or to an allogeneic recipient or host) and reintegrated into the systemic circulation using standard microsurgical techniques. Also known as “microvascular free flaps” or “microvascular free tissue,” these microvascular beds can support skin, bone, muscle or adipose tissue and are used clinically thousands of times each year in reconstructive surgery (Gurtner et al., Plast Reconstr Surg; 106:672-82; quiz 683 (2000)). They are employed to reconstruct ablative, congenital or traumatic defects in humans.

According to the present disclosure, a tissue of interest is harvested as an explant and subsequent reattachment or reanastomosis. The tissue of interest may be a microvascular bed or microvascular “free flap”. A microvascular bed or free flap is an intact microcirculatory network or bed. Microvascular free flap transfer is the auto-transplantation of composite tissues (known as a free flap) from one anatomic region to another (Blackwell et al., Head Neck; 19:620-28 (1997)). Clinically, it is routinely performed to reconstruct defects following tumor extirpation such as in a mastectomy. In performing microvascular free flap transfer, an intact microcirculatory network or bed is detached.

A pedicled flap may use a piece of skin and underlying tissue that lie adjacent to the wound. The flap remains attached at one end so that it continues to be nourished by its original blood supply, and is repositioned over the wounded area. A regional flap uses a section of tissue that is attached by a specific blood vessel. When the flap is lifted, it needs only a very narrow attachment to the original site to receive its nourishing blood supply from the tethered artery and vein.

A musculocutaneous flap, also called a muscle and skin flap, is used when the area to be covered needs more bulk and a more robust blood supply. Musculcutaneous flaps are often used in breast reconstruction to rebuild a breast after mastectomy. This type of flap remains “tethered” to its original blood supply. In a bone/soft tissue flap, bone, along with the overlying skin, is transferred to the wounded area, carrying its own blood supply.

In some aspects, methods and compositions are provided for treating or preventing ischemic injury in a skin flap in a subject.

In some aspects, methods and compositions are provided for treating or preventing ischemic injury in a fascia flap in a subject.

In some aspects, methods and compositions are provided for treating or preventing ischemic injury in a muscle flap in a subject.

In some aspects, the tissue flap is autologous relative to a tissue flap recipient.

In further aspects, the tissue flap is heterologous relative to a tissue flap recipient.

### Tissue Flap Harvesting

Microvascular free flap transfer generally entails the division and subsequent re-anastomosis of the dominant artery and vein in the composite tissue (flap), allowing the transplanted tissue to survive. As such, microvascular free tissue transfer represents the manipulation and transfer of an intact microcirculatory network or bed. This network can supply a variety of tissues because of its functioning microcirculatory network. This vascular network may be detached from the intact organism and maintained ex vivo, permitting its manipulation or modification without danger of systemic toxicity.

When in their normal, native state, microvascular beds contain all of the distinct, constituent cells that exist within the microcirculation (Krapohl et al., Plast Reconstr Surg; 102:2388-94 (1998); Taylor et al., Br J Plast Surg; 40:113-41 (1987)). Grossly, they consist of large muscular arteries, leading to capacitance arterioles, endothelial lined capillaries, venules, veins and all of the phenotypically distinct cells within them (Siemionow et al., Ann Plast Surg; 41:275-82 (1998); Carroll et al., Head Neck; 22:700-13 (2002)). Importantly, in the native state, they contain all of these cell types in a functional and precisely ordered three-dimensional configuration. In a sense, they have already been “patterned”. These expendable microvascular beds provide an ideal, living substrate on which to fabricate a “neo-organ,” i.e., a non-naturally occurring vascularized tissue that provides a function of a gland or organ, or that supplements the function of a gland or organ. Since microvascular free flaps contain a single afferent artery and efferent vein they can be easily reintegrated into the systemic circulation by standard vascular anastamoses.

According to the present disclosure, a selected tissue may be excised (“harvested”) by conventional surgical methods known in the art (see, e.g., Petry et al., Plast Reconstr Surg; 74:410-13 (1984); Blackwell et al., Head Neck; 19:620-28 (1997)). In the case of a skin flap, the surgical procedure results in the removal of skin and subcutaneous tissue associated with blood vessels in a select region of the body.

The microvascular tissue flaps may comprise tissue that includes, but is not limited to, epithelial tissues, e.g., the epidermis, gastrointestinal tissue; connective tissues, e.g., dermis, tendons, ligaments, cartilage, bone and fat tissues, blood; muscle tissues, e.g., heart and skeletal muscles; nerve tissue, e.g., neurons and glial cells. The microvascular free flaps or beds can also comprise tissue derived from organs or organ systems such as the skeletal system, e.g., bones, cartilage, tendons and ligaments; the muscular system, e.g., smooth and skeletal muscles; the circulatory system, e.g., heart, blood vessels, endothelial cells; the nervous system, e.g., brain, spinal cord and peripheral nerves; the respiratory system, e.g., nose, trachea and lungs; the digestive system, e.g., mouth, esophagus, stomach, small and large intestines; the excretory system, e.g., kidneys, ureters, bladder and urethra; the endocrine system, e.g., hypothalamus, pituitary, thyroid, pancreas and adrenal glands; the reproductive system, e.g., ovaries, oviducts, uterus, vagina, mammary glands, testes, seminal vesicles and penis; the lymphatic and immune systems, e.g., lymph, lymph nodes and vessels, white blood cells, bone marrow, T- and B-cells, macrophage/monocytes, adipoctyes, keratinocytes, pericytes, and reticular cells.

In another aspect of the present disclosure, a composite tissue flap, i.e., a flap composed of bone and skin, muscle and skin, adipose tissue and skin, fascia and muscle, or other such combination known to normally be present in the mammal body, is used because it has a greater tolerance for ischemia.

In certain aspects, the selected tissue is autologous. In other aspects, the tissue is heterologous.

Once the flap is excised, the proximal blood vessels that are associated with the flap are clamped while the flap is ex vivo. Any conventional technique known in the art can be used to clamp the blood vessels.

The selected tissue is maintained ex vivo by methods for maintaining explants well-known in the art. The tissue is preferably perfused, e.g., the tissue can be wrapped in gauze, a catheter can be placed in a blood vessel associated with the tissue and secured with a suture, and the tissue perfused or infused with physiological saline. In one aspect, the perfusion is conducted at a cold temperature (for cold ischemia). In other aspects, perfusion is conducted at room temperature or body temperature. Preferably, the tissue is perfused ex vivo through a catheter at a constant perfusion pressure to flush out blood from the flap vessels. Preferably, the infusions are given at physiologic pressures (80-200 mm Hg), since high pressures cause excessive tissue damage, leading to necrosis of all or part of the flap. A continuous microperfusion system, such as the one described by Milas et al. (Clinical Cancer Research; 3(12-1):2197-2203 (1997)) may be used.

Tissue can survive ex vivo for a short time (i.e., hours) with no significant effect on vascular patency and cellular function following re-implantation. Longer periods of ex vivo maintenance may, in some instances cause microvascular flap failure (i.e., thrombosis, endothelial damage, and/or edema). These conditions are assessed by the clinical judgment of the ordinarily skilled practitioner, as well as by, e.g., histological evaluation with standard histological sections taken from both proximal, middle, and distal microvascular bed and surrounding normal tissue.

### Methods of Tissue Reimplantation

Using conventional surgical procedures (see e.g., Petry et al., Plast Reconstr Surg; 74:410-33 (1984); Blackwell et al., Head Neck; 19:620-28 (1997)), the flap is then reinserted into the patient and re-anastomosed to a section of the circulatory system in the patient. Preferably, the flap is attached non-orthotopically, i.e., it is re-anastomosed to a different area of the patient's circulatory system. For example, a flap may be detached from its supply from the femoral artery and then transplanted to the region of the carotid artery and attached to the carotid arterial system. In another aspect, the flap is reattached to the blood vessels from which it was excised. Preferably, a splint or other protective device is placed over the operative site after attachment or re-anastomosis.

In certain cases, re-implantation of the microvascular free flap may produce a substantial degree of scarring, thus obscuring the viability of the tissue independent from surrounding tissue. If this occurs, methods commonly known in the art, such as separation with silicone sheets, may be utilized to separate a re-implanted microvascular free flap from the host in order to prevent tissue ingrowth.

### Therapeutic Regimens for Head and Neck Cancers

Patients with recurrent or locally metastatic head and neck cancers present unique challenges to the head and neck surgeon. Head and neck tumors are characterized by a significant degree of morbidity and mortality caused in large part by local tumor extension and invasion. One particularly aggressive and common form of head and neck tumor is head and neck squamous cell carcinoma (SCC). SCC tumors, accounting for 6% of all new cancers in this country and 12,500 deaths each year (Landis et al., Cancer J Clin; 1:6-29 (1998)), are particularly difficult to obtain local control following surgery. The head and neck surgeon is frequently involved in the care of these patients, often in combination with the reconstructive plastic surgeon. This inter-disciplinary care has resulted in advancements in surgical ablative techniques as well as the availability of novel reconstructive modalities. However, despite more aggressive surgery (made possible in part by the availability of microsurgical reconstruction) as well as novel radiologic and chemotherapeutic approaches, the mortality rates for this population of tumors have not significantly improved during the last 30 years (Vokes et al., N Engl J Med; 328:184-191 (1993)). This disappointing reality highlights the need for novel therapeutic approaches for head and neck SCC.

The advent of reconstructive microsurgery, however, has greatly aided the care of the oncologic head and neck patient. Free tissue transfer is now routinely used to close defects that were not amenable to closure several decades ago, and has improved the care of the head and neck patient by enabling improved surgical palliation, such as adequate oral continence following removal of a tumor of the mouth. In addition, wider resections are now routinely carried out due to the availability of reliable reconstructive options.

Free tissue transfers have mostly been used for closure of defects (i.e., as fillers) and to enable some return of function (e.g., in the restoration of a competent oral sphincter or space esophageal tube).

Oral cancer is a serious malignant disease which is fatal if not treated. With more radical ablation for oral cancer, obtaining a good aesthetic appearance and good function after surgical reconstruction has become increasingly difficult. The use of a regional flap is still popular in head and neck reconstruction. Since its introduction by Bakamjian (Bakamjian, V. Y.; Plast Reconstr Surg; 36:173-84 (1965)) in 1965, the medially based deltopectoral (DP) flap has become the premier flap in head and neck surgery.

Multiple flap types are available to surgeons when conducting oral reconstruction. For instance, the DP flap is a fasciocutaneous flap that is composed of fascia, subcutaneous tissue, and skin. The DP flap is a direct cutaneous axial flap supplied by the anterior thoracic perforators of the internal mammary artery for the first four intercostal spaces. In 1979, Ariyan (Ariyan S., Plast Reconstr Surg; 63(1):73-81 (1979)) introduced the pedicled pectoralis major myocutaneous (PMMC) flap. The PMMC flap is made of muscle and subcutaneous fat.

Free flaps have been common options for reconstruction in the head and neck region since the 1980s (Peterson (editor), Principle of oral and maxillofacial surgery. Philadelphia: J B Lippincott; 1015-104 (1992) (citing Rohrich, et al., The use of free tissue transfer in head and neck reconstruction (1992)). The free flap, with its rich vascularity, permits a high degree of versatility and reliability in design and is a useful reconstruction method for postoperative defects. Using a free flap as well as a pedicled flap, reconstructions can be performed individually.

### Therapeutic Regimes for Skin Cancer

With a substantial rise in the incidence of skin cancer, skin surgery, and flap surgery in particular, has become increasingly common. Substantial increases in the incidence of skin cancer are found in all three of the most common types of skin cancers, namely, basal cell carcinoma (BCC), squamous cell carcinoma (SCC), and malignant melanoma (MM). Simple closure is not possible in approximately 10% of excisions. In the remaining patients, skin flaps or skin grafts are necessary. In most cases, flaps compared with skin grafts offer the best cosmetic end result. Local flaps are often classified according to movement into transposition, advancement, and rotation flaps (American Academy of Dermatology, J Am Acad Dermatol; 34:703-8. 8601669 (1996)).

### Therapeutic Regimes for Mastectomies

The current treatment of breast cancer includes surgery, chemotherapy and radiation therapy, and combinations of these three modalities. Approximately one-half of the women in the U.S. that are diagnosed with breast cancer will elect or will require a mastectomy.

Closure of the skin defect created by a mastectomy may involve the immediate or delayed incorporation of a cutaneous or myocutaneous tissue flap to at least partially replace the excised tissue. Myocutaneous units are commonly used to cover defects, whether traumatic or post-resectional. Myocutaneous units are prepared as a combination of both skin and muscle, or as a muscle units that subsequently are skin grafted. Myocutaneous units are transferred as free flaps (flaps detached from intrinsic blood supply), thereafter connecting the unit's axial blood supply to recipient vessels near the defect.

Latissimus dorsi or rectus abdominis myocutaneous flaps were the most frequently utilized myocutaneous flaps for post-mastectomy closure. Some common closure applications for latissimus dorsi flaps include coverage of defects in the head and neck area, especially defects created from major head and neck cancer resection; additional applications include coverage of chest wall defects other than mastectomy deformities. The latissimus dorsi was also used as a reverse flap, based upon its lumbar perforators, to close congenital defects of the spine such as spina bifida or meningomyelocele.

Due to the adverse characteristics of a mastectomy deformity, either from a radical mastectomy or a modified radical mastectomy, many women opt for post-mastectomy breast reconstruction. Reconstruction can take place contemporaneously with the mastectomy, or at a later time.

To achieve breast reconstruction, it is common to use a submuscular breast expander or a permanent implant in conjunction with some form of a mastectomy closure technique. A breast expander allows for, and generally requires, sequential addition of fluid to stretch the remaining breast tissue. Accordingly, expanders or implants (“breast inserts”) are beneath the mastectomy incision, and have been used as a method for either immediate or delayed breast reconstruction.

There are several disadvantages to post-mastectomy use of former myocutaneous flaps, in the context of excision closure or of post-surgical breast reconstruction. In either of these contexts, most procedures cause a significant transverse scar across the chest. The donor site scar on the back is also substantial. When such procedures are used and a breast is reconstructed, the disadvantages are exacerbated since there is a large elliptical paddle of skin across the breast. This skin paddle has different pigmentation than the adjacent breast skin. Furthermore, the large flap of skin does not adequately recreate the contour of the breast.

Tissue flaps used for breast reconstruction may be a cutaneous flap which comprises cutaneous tissue, subcutaneous tissue and inherent circulatory vessels; or, a myocutaneous flap which comprises muscular tissue, cutaneous tissue, subcutaneous tissue and inherent circulatory vessels. As used herein, cutaneous is defined to mean a fully epithelialized or a partially deepithelialized flap. The flap may be a free flap or a pedicled flap where the inherent vessels remain connected with the native blood supply. In the case of pedicled flaps, the blood supply to the flap is kept intact and moved with the flap. In the case of a free flap, the flap may be detached and reattached at the chest wall site by vascular techniques known in the art.

Reconstruction may be a post-mastectomy procedure, a post-traumatic procedure, or a procedure done to enlarge or decrease the volume of the breast. A reconstruction may be contemporaneous with a mastectomy or may be delayed, taking place over one or more post-mastectomy surgical procedures.

In accordance with the present disclosure, a delayed procedure comprises: a multistage procedure where a mastectomy is performed with contemporaneous placement of an expander, and a subsequent procedure when a tissue flap reconstruction is performed; a mastectomy; a subsequent procedure when an expander is placed, and a subsequent procedure when a tissue flap reconstruction is performed; revisions to a previous reconstruction; or, the placing or modifying of breast implant materials.

An immediate reconstruction procedure in accordance with the present disclosure may comprise the use of a latissimus dorsi muscle following a modified radical circumareolar mastectomy. Immediate reconstruction may also be employed with autologous sources other than the latissimus dorsi.

### Treatment and Prevention of Ischemia in Tissue Flaps by APC

Tissue flap survival following surgical procedures, especially reconstructive surgical procedures, is often compromised by, among other complications, infection, ischemia and tissue edema. Inflammation is the body's reaction to injury and infection. Three major events are involved in inflammation: (1) increased blood supply to the injured or infected area; (2) increased capillary permeability enabled by retraction of endothelial cells; and (3) migration of leukocytes out of the capillaries and into the surrounding tissue (i.e., cellular infiltration) (Roitt et al., Immunology; (1989)).

Tissue and skin flap breakdown remain a major problem in reconstructive surgery, especially in patients suffering from diabetic microangiopathy or other forms of peripheral vascular disease. In such patients wound healing is often delayed and defective and in these patients complications may lead to necrosis and eventually require costly and painful secondary surgical procedures.

The present disclosure discloses the first demonstration of the benefit of APC in critical ischemia. Three systemic APC injections significantly improve the survival of ischemic flaps a week after injection. Although a single dose of APC is efficacious in reducing ischemia-reperfusion injury acutely in animal models, given the short half-life of APC, it is speculated that such an approach would be inadequate in the setting of sustained ischemia (Yamaguchi et al., Hepatology; 25:1136 (1997); Mizutani et al., Blood; 95:3781 (2000); Schoots et al., Crit Care Med; 32:1375 (2004); Dillon et al., J Orthop Res; 23:1454 (2005); Hoffmann et al., Crit Care Med; 32:1011 (2004)). Although informative, the clinical applicability of these previous ischemia-reperfusion injury studies is limited by the use of supraphysiologic doses of APC as much as a thousand-fold higher than those safely employed in human subjects (Ercocen et al., Dermatol Surg; 29:692 (2003)). By contrast, the present disclosure discloses APC doses corresponding to the hourly dose in septic patients treated with the drug (Bernard et al., N Engl J Med; 344:699 (2001)). Even at this clinically relevant dose, as seen in the late preoperative group and reported in several human sepsis trials, the major risk associated with APC is increased bleeding (Bernard et al., N Engl J Med; 344:699 (2001); Fumagalli et al., Crit Care; 11 Suppl 5:S6 (2007)). This increased bleeding can be avoided by either postponing APC administration until after flap elevation or performing it well in advance of surgery. Both of these approaches result in significantly improved flap survival as compared to control animals. In fact, near-complete flap survival results from early preoperative APC administration. Because the half-life of APC is short, its anticoagulant activity drops off within a short time enabling it to be administered shortly before a surgical procedure (Hoffmann et al., Crit Care Med; 32:1011 (2004)). For this reason, it may be supposed that APC's benefit results from its cytoprotective activities in the immediate perioperative period. Recently, Kerschen et al. demonstrated that an APC variant lacking anticoagulant activity is still effective in treating sepsis (Kerschen et al., J Exp Med; 204:2439 (2007)). Non-anticoagulant APC may also be effective in improving flap survival, thereby broadening the clinical indications for the agent.

The histologic assessment in Example 4 further supports theories of cytoprotective and pro-angiogenic roles for APC by showing improved striated muscle viability and increased capillary numbers in APC treated compared to control or untreated animals. The real-time PCR analysis in Example 6, suggests a potential mechanism by which APC could induce new vessel formation and growth by modulating pro-angiogenic factors. In keeping with previous evidence, a significant early upregulation in mRNA levels of the transcription factor Egr-1 is observed following flap elevation (Asano et al., Circ J; 71:405 (2007)). However, this upregulation was significantly augmented by APC injection. Recently, APC has been shown to induce Egr-1 in vascular endothelium via an EPCR-independent pathway. Egr-1 signaling in turn protects striated muscle and endothelial cells from apoptosis (Asano et al., Circ J; 71:405 (2007); O'Brien et al., Arterioscler Thromb Vasc Biol; 27:2634 (2007)). In the case of endothelial cells, cytoprotection involves suppression of the pro-apoptotic mediator TNF-related apoptosis-inducing ligand (TRAIL) (O'Brien et al., Arterioscler Thromb Vasc Biol; 27:2634 (2007)). Egr-1 is now held to be a master regulator in angiogenesis, leading to the downstream upregulation of factors such as fibroblast growth factor-2 and tissue factor, and VEGF (Schweighofer et al., Clin Hemorheol Microcir; 37:57 (2007); Lucerna et al., J Biol Chem; 278:11433 (2003); Fahmy et al., Nat Med; 9:1026 (2003)). Although no differences have been identified between APC and control animals with regards to VEGF transcription levels, higher levels of VEGFR2 are noted when APC is administered. Previously Chen et al. demonstrated that ischemia induces VEGFR2 in a muscle flap model and it can be speculated that APC preferentially upregulates VEGFR2, but not its ligand (Chen et al., J Surg Res; 140:45 (2007)). It remains unclear whether this pattern is mediated through Egr-1. The observed pattern in Bcl-2 transcription is in keeping with previous studies supporting APC's anti-apoptotic activity (Cheng et al., Nat Med; 9:338 (2003); Mosnier et al., Biochem J; 373:65 (2003)).

Although pro-angiogenic factors are expressed primarily in endothelial cells, the source cell types of the pro-inflammatory gene transcripts in the biopsy specimens cannot be as clearly identified. The early downregulation of ICAM-1 observed is likely due to direct APC signaling on endothelial cells. This early decrease in ICAM-1 in turn leads to decreased neutrophil trafficking into the flap tissue and the dramatic decrease in TNF-α observed 24 hours postoperatively in APC-treated animals. The inhibition of NF-κB in endothelial cells by APC likely plays a lesser role in the decrease in TNF-α (Joyce et al., Crit Care Med; 30:S288 (2002)). The lack of difference in IL-1β transcription between APC and control groups is not altogether surprising as IL-1β regulation is primarily post-translational (Burns et al., Curr Opin Immunol; 15:26 (2003)).

The observation that early gene expression changes in the immediate perioperative period correlated with histological changes days after surgery is novel. This suggests that APC's benefits stem more from its immediate cytoprotective effects than its anticoagulant and pro-angiogenic activities. Although these latter properties are contributory, they are likely secondary to APC's reduction of pro-inflammatory mediators and its anti-apoptotic effects on the endothelium in the critical hours following surgery. By preserving the existing microcirculation through its early cytoprotective activities, APC buys time for new vessel formation and improved flap survival days after administration.

Systemic injection of APC significantly improves the survival of ischemic cutaneous flaps, seemingly by inducing cytoprotective and angiogenic pathways. Unlike previous single-target pharmacologic therapies, the present approach appears to offer the benefits of surgical delay without additional operative morbidity. In this way, APC shows considerable promise as a therapeutic agent in the field of reconstructive surgery.

### Therapeutic Compositions

Compositions for use in accordance with the present disclosure may be formulated in a conventional manner using one or more physiologically acceptable carriers comprising excipients and auxiliaries which facilitate processing of a therapeutic composition into preparations which can be used pharmaceutically. These therapeutic compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen.

In certain aspects, a therapeutic composition is provided comprising one or more of: (i) an activated protein C (APC), (ii) a functional fragment of an APC, (iii) an APC mimetic compound, and (iv) a derivative of APC; optionally in admixture with a pharmaceutically acceptable carrier or additive.

In further aspects, a therapeutic composition is provided comprising one or more of: (i) an activated protein C (APC), (ii) a functional fragment of an APC, (iii) an APC mimetic compound, and (iv) a derivative of APC; optionally in admixture with a pharmaceutically acceptable carrier or additive, wherein the composition treats or prevents ischemic injury in a tissue flap in a subject.

The therapeutic compositions are generally formulated as sterile, substantially isotonic and in full compliance with all Good Manufacturing Practice (GMP) regulations of the U.S. Food and Drug Administration. From the foregoing description, various modifications and changes in the compositions and methods will occur to those skilled in the art. All such modifications coming within the scope of the appended claims are intended to be included therein. Each recited range includes all combinations and sub-combinations of ranges, as well as specific numerals contained therein.

When a therapeutically effective amount of a composition of the present method is administered by e.g., intradermal, cutaneous or subcutaneous injection, the composition is preferably in the form of a pyrogen-free, parenterally acceptable aqueous solution. The preparation of such parenterally acceptable protein or polynucleotide solutions, having due regard to pH, isotonicity, stability, and the like, is within the skill in the art. A preferred composition should contain, in addition to protein or other active ingredients of the present disclosure, an isotonic vehicle such as Sodium Chloride Injection, Ringer's Injection, Dextrose Injection, Dextrose and Sodium Chloride Injection, Lactated Ringer's Injection, or other vehicle as known in the art. The composition of the present disclosure may also contain stabilizers, preservatives, buffers, antioxidants, or other additives known to those of skill in the art. The agents of the present disclosure may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compositions can be formulated readily by combining the active compounds with pharmaceutically acceptable carriers well known in the art. Such carriers enable the compounds of the present disclosure to be formulated as tablets, pills, dragees, powders, capsules, liquids, solutions, gels, syrups, slurries, suspensions and the like, for oral ingestion by a patient to be treated.

Therapeutic compositions for parenteral administration include aqueous solutions of the compositions in water-soluble form. Optionally, the suspension may also contain suitable stabilizers or agents which increase the solubility of the compositions to allow for the preparation of highly concentrated solutions. Alternatively, the active ingredient may be in powder form for constitution with a suitable vehicle, e.g., sterile pyrogen-free water, before use.

In general, enteral dosage forms for the therapeutic delivery of polypeptides are less effective because in order for such a formulation to be efficacious, the peptide must be protected from the enzymatic environment of the gastrointestinal tract. Additionally, the polypeptide must be formulated such that it is readily absorbed by the epithelial cell barrier in sufficient concentrations to effect a therapeutic outcome. The polypeptides of the present method may be formulated with uptake or absorption enhancers to increase their efficacy. Such enhancers include for example, salicylate, glycocholate/linoleate, glycholate, aprotinin, bacitracin, SDS caprate and the like. An additional detailed discussion of oral formulations of peptides for therapeutic delivery is found in Fix, J Pharm Sci; 85(12):1282-1285 (1996), and Oliyai, et al., Ann Rev Pharmacol Toxicol; 32:521-544 (1993), this aspect of these two references is incorporated herein by reference.

In further compositions, proteins or other active ingredients of the present method may be combined with other agents beneficial to the treatment of tissue flaps.

### Methods of Administration

Therapeutic compositions of the present method may be administered by any known route. By way of example, the composition may be administered by a mucosal or other localized or systemic route (e.g., enteral and parenteral). In particular, achieving a therapeutically effective amount of activated protein C, prodrug, or functional variant in the body may be desired.

“Parenteral” includes subcutaneous, intradermal, intramuscular, intravenous, intra-arterial, intrathecal, and other injection or infusion techniques, without limitation.

In various aspects, the agent is administered systemically.

In some aspects, the agent is administered parenterally.

In some aspects, the agent is administered intravenously.

Suitable choices in amounts and timing of doses, formulation, and routes of administration can be made with the goals of achieving a favorable response in the subject (i.e., efficacy or therapeutic response), and avoiding undue toxicity or other harm thereto (i.e., safety). Administration may be by bolus or by continuous infusion. Bolus refers to administration of a drug (e.g., by injection) in a defined quantity (called a bolus) over a period of time. Continuous infusion refers to continuing substantially uninterrupted the introduction of a solution into a blood vessel for a specified period of time.

In some aspects, the agent is administered through continuous infusion.

In some aspects, the agent is administered as a bolus.

APC may be administered before tissue flap surgery, after tissue flap surgery, or both before and after surgery. Since APC has anticoagulant activity, it is not ideally administered during any surgical procedure. Because APC has a half-life of approximately 20 minutes, however, it would be present at only negligible levels after three hours (Hoffmann et al., Crit Care Med; 32:1011 (2004)). According to the present disclosure, APC should be administered a sufficient amount of time before and/or after tissue flap surgery, such that the risk of hemorrhage is minimal.

In various aspects, the agent is administered to a subject pre-surgery.

In further aspects, the agent is administered to a subject pre-surgery and post-surgery.

In some aspects, the agent is administered to a subject more than one hour pre-surgery.

In various aspects, the agent is administered to a subject a sufficient amount of time pre-surgery such that the risk of hemorrhage during surgery is minimal.

In some aspects, the agent is administered to a subject more than one hour post-surgery.

In some aspects, the agent is administered to a subject a sufficient amount of time post-surgery such that the risk of hemorrhage following surgery is minimal.

Treatment may involve a continuous infusion (e.g., for 3 hr) or a slow infusion (e.g., for 24 hr to 72 hr). Alternatively, it may be administered daily, every other day, once a week, or once a month. Dosage levels of active ingredients in a therapeutic composition may also be varied so as to achieve a transient or sustained concentration of the agent or derivative thereof in a subject and to result in the desired therapeutic response.

Thus, “effective” refers to such choices that involve routine manipulation of conditions to achieve a desired effect (e.g., inhibition of apoptosis or cell death, promotion of cell survival, cytoprotection, neuroprotection, or combinations thereof). The amount of APC administered in a bolus may be from 0.005 μg to 2000 μg per kg of body weight, or more preferably between 0. 1 to 40 μg per kg of body weight, depending upon a particular subject's therapeutic needs.

The therapeutic amount may be based on titering to a blood level amount of APC of about 0.01 μg/ml to about 1.6 μg/ml, preferably from about 0.01 μg/ml to about 0.5 μg/ml. It is also within the skill of the art to start doses at levels lower than required to achieve the desired therapeutic effect and to gradually increase the dosage until the desired effect is achieved. It is likewise within the skill of the art to determine optimal concentrations of APC to achieve the desired effects of the present disclosure, e.g., about 1-100 nM.

Those of skill in the art will be able to modify and adjust these techniques according to the therapeutic needs of a subject undergoing reconstructive surgery and treatment with APC.

### Kits

The present disclosure also provides kits comprising one or more containers of compositions of the present disclosure. Compositions can be in liquid form or can be lyophilized. Suitable containers for the compositions include, for example, bottles, vials, syringes, and test tubes. Containers can be formed from a variety of materials, including glass or plastic. A container can have a sterile access port (for example, the container can be an intravenous solution bag or a vial having a stopper pierceable by a hypodermic injection needle).

The kit can further comprise a second container comprising a pharmaceutically acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device(s). The kit can further include a third component comprising an adjuvant.

The kit can also comprise a package insert containing written instructions for methods of administering the compositions of the present disclosure or methods of treating or preventing ischemic injury in a tissue flap. The package insert can be an unapproved draft package insert or can be a package insert approved by the Food and Drug Administration (FDA) or other regulatory body.

The present disclosure also provides a delivery device pre-filled with the compositions of the present disclosure.

In certain aspects, a kit is provided comprising one or more of: (i) an activated protein C (APC), (ii) a functional fragment of an APC, (iii) an APC mimetic compound, and (iv) a derivative of APC; optionally in admixture with a pharmaceutically acceptable carrier or additive.

In further aspects, a kit is provided comprising one or more of: (i) an activated protein C (APC), (ii) a functional fragment of an APC, (iii) an APC mimetic compound, and (iv) a derivative of APC; optionally in admixture with a pharmaceutically acceptable carrier or additive, wherein the kit is used to treat or prevent ischemic injury in a tissue flap in a subject.

Although the foregoing methods and compositions have been described in detail by way of example for purposes of clarity of understanding, it will be apparent to the artisan that certain changes and modifications are comprehended by the disclosure and can be practiced without undue experimentation within the scope of the appended claims, which are presented by way of illustration not limitation.

### EXEMPLARY ASPECTS

### Example 1

### Rat Ischemic Skin Flap Model

Ethics approval for the study was obtained from the University Committee on Laboratory Animals, Dalhousie University. All experiments were performed in accordance with the Guide to the Care and Use of Experimental Animals of the Canadian Council on Animal Care. A total of 44 adult male Sprague-Dawley rats (Charles River Laboratories, Montreal, QC) weighing an average of 562±11 grams were randomly assigned into control and APC groups. All animals were separately housed with a light/dark cycle of 12 hours and provided chow and water ad libitum. Anaesthesia was induced by intraperitoneal sodium pentobarbital injection (25 mg/kg; McGill University, Montreal, QC) and maintained with inhaled 2% isoflurane (Baxter Co., Toronto, ON). After shaving of the dorsal hair, a cranially based dorsal cutaneous flap (3×7 cm), beginning 1 cm below the scapular angle, was undermined and elevated as previously described (Zhi et al., Plast Reconstr Surg; 120:1148 (2007)). The flap was immediately returned to its bed using 4-0 monofilament nylon (Surgilon; Synetec, Norwalk, Conn.). All animals received a subcutaneous injection of buprenorphine (0.03 mg/kg; McGill University, Montreal, QC) following wound closure.

### Example 2

### Activated Protein C Injection Preparation and Timing

The APC solution was formed by dissolving 0.5 mg of recombinant human APC powder (Sigma Chemical Co., St. Louis, Mo.) in ice-cold phosphate-buffered saline (pH=7.2) to a final concentration of 12.5 μg/ml. Each experimental animal subsequently received 25 μg/kg of APC via tail vein injection, while control animals received an equal volume of phosphate-buffered saline per injection.

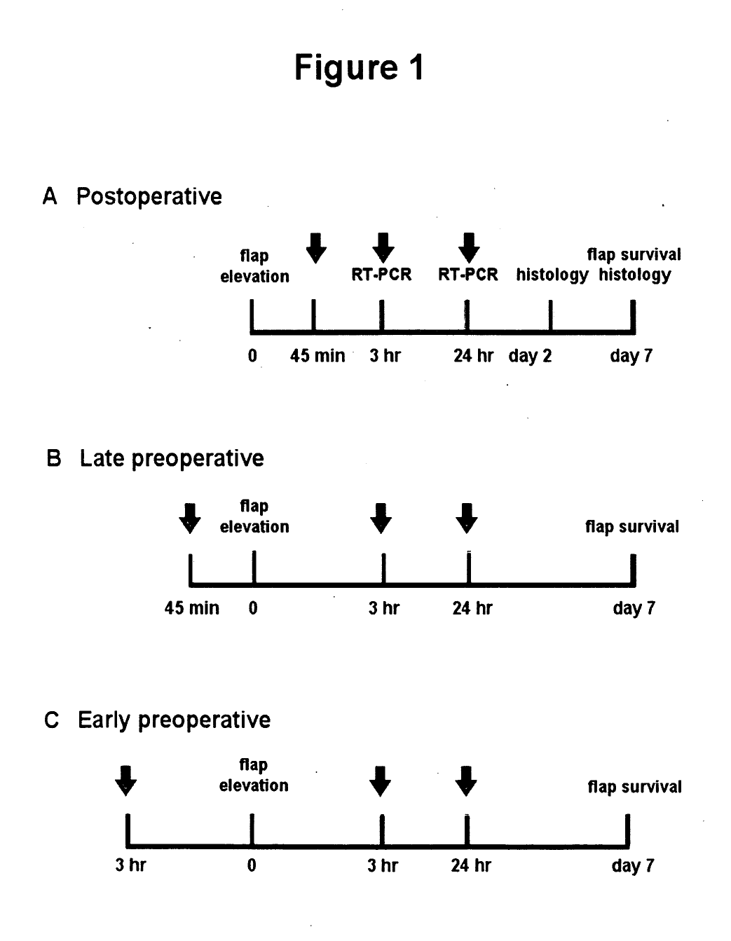

Animals were divided into three experimental groups (FIG. 1): postoperative (n=12 APC-treated or control animals); late preoperative (n=5); and early preoperative (n=5). For each experimental group, animals were randomly assigned to receive either APC or PBS injections. Three tail vein injections were performed per animal. For the postoperative group, the first injections were performed 45 minutes following flap elevation. Subsequent histologic and RT-PCR analyses were performed using this experimental group. For the late preoperative group, the first injections were performed 45 minutes prior to surgery. For the early preoperative group, the first injections were performed 3 hours prior to flap elevation. For all animals in these three groups, the second and third injections were performed at 3 and 24 hours following surgery, respectively. These additional time-points were selected as major transcriptional changes have been shown to occur at them during flap ischemia (Chen et al., J Surg Res; 140:45 (2007); Zhang et al., J Reconstr Microsurg; 22:641 (2006); Michlits et al., Wound Repair Regen; 15:360 (2007); Huang et al., Circ J; 70:1070 (2006)).

### Example 3

### Measurement of Flap Survival Rate

Flap survival rate was observed on postoperative day 7. Rats were euthanized with intraperitoneal sodium pentobarbital and photographs taken with a digital camera (Powershot A5; Canon, Tokyo, Japan). Zones of dark color or covered with scabs were defined as necrotic, while remaining areas were defined as viable. To assess survival rate, the digital image was processed using image analysis software (Photoshop CS2; Adobe Systems Inc., San Jose, Calif.). The total and viable areas of each flap were measured and survival rate expressed as a percentage of the total flap area (survival rate=viable area/total area×100%).

### Systemic APC Treatment Improves Flap Survival

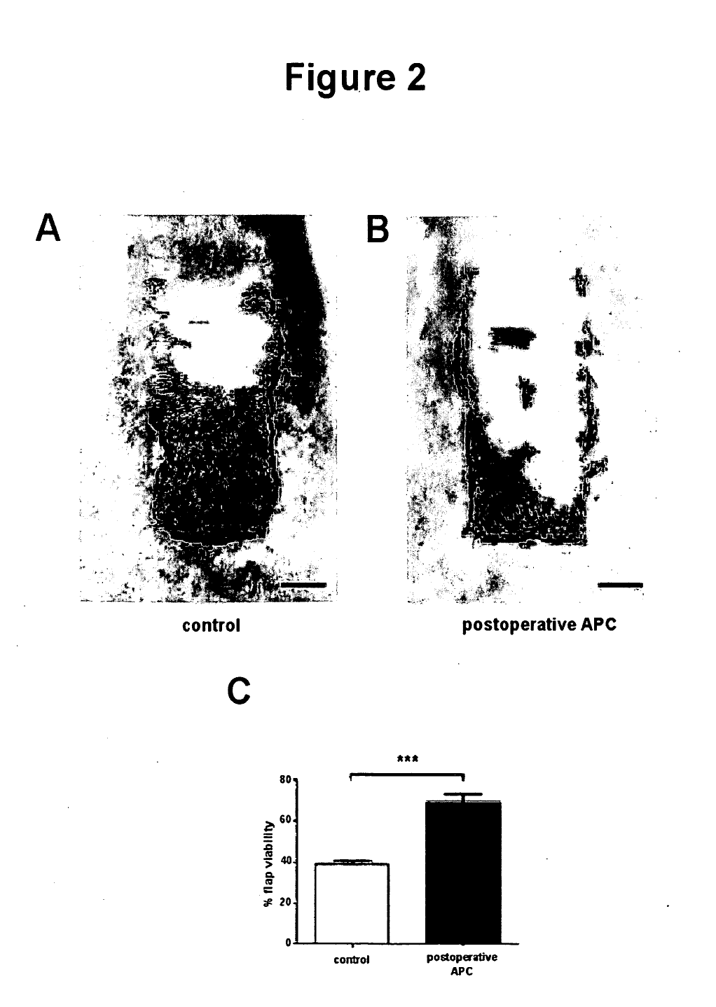

APC is a cytoprotective agent that has beneficial effects in animal models of ischemia-reperfusion injury (Yamaguchi et al., Hepatology; 25:1136 (1997); Mizutani et al., Blood; 95:3781 (2000); Schoots et al., Crit Care Med; 32:1375 (2004); Dillon et al., J Orthop Res; 23:1454 (2005)). APC was evaluated to see if similar beneficial effects could be observed in a rat model of critical flap ischemia. Since APC has anticoagulant activity, in the initial experimental group, APC administration was delayed until after flap elevation. In this postoperative group, no increased bleeding or hemorrhage was noted during flap elevation or over the subsequent week. As shown in FIG. 2, APC injection resulted in significantly improved flap survival compared to PBS injection (68.9±4.3 percent for APC group versus 39.3±1.5 percent for control group; p<0.001).

### Example 4

### Histologic Assessment

Full-thickness 3 mm punch (Miltex, York, Pa.) biopsies were performed on postoperative days 2 or 7 (n=3, per group, per time-point). Biopsies were taken from the flap's midline, 3 cm proximal to its caudal end. Samples were fixed in 4% paraformaldehyde for 24 hours, followed by immersion in 70% ethanol at 4° C. for 24 to 48 hours. Samples were subsequently embedded in paraffin and 5-μm-thick sequential sections were cut. Sections were stained with hematoxylin and eosin. Day 7 biopsy sections underwent periodic acid Schiff (PAS) staining to visualize the basement membranes of blood vessels as well as immunostaining with rabbit polyclonal antibody against rat factor VIII-related antigen (DAKO, Carpinteria, Calif.) as described (Villaschi et al., Lab Invest; 71:299 (1994); McManus, J. F., Biotechnic Histochem; 23:99 (1948)). Specimens were examined under light microscopy by a blinded hematopathologist. Quantification of polymorphonuclear cells (PMNs) and the total percentage of viable muscle fibers in the panniculus carnosus were performed under 40× magnification in three sections taken from each day 2 biopsy as previously described (Kim et al., Plast Reconstr Surg; 120:1774 (2007); Zhang et al., J Invest Dermatol; Epub ahead of print (2008)). To assess the effect of APC on angiogenesis, the average number of blood vessels in the hypodermis per high-power field (HPF) was determined for each day 7 section. To ensure that observed differences in blood vessels were not an indirect effect of increased overall flap survival, these values were compared to the number of vessels present in skin biopsies taken from normal rats that had neither undergone surgery nor APC/PBS injection.

### Early and Late Histologic Changes Following APC Treatment

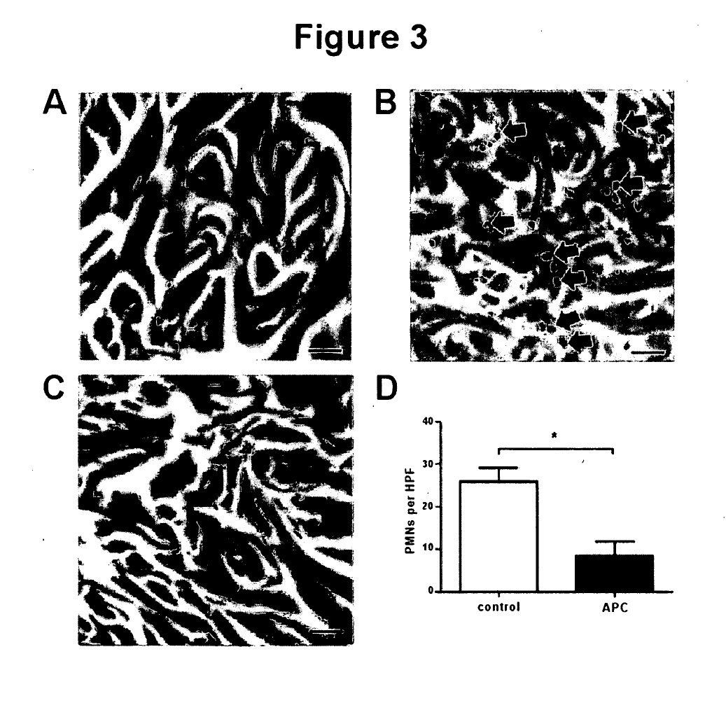

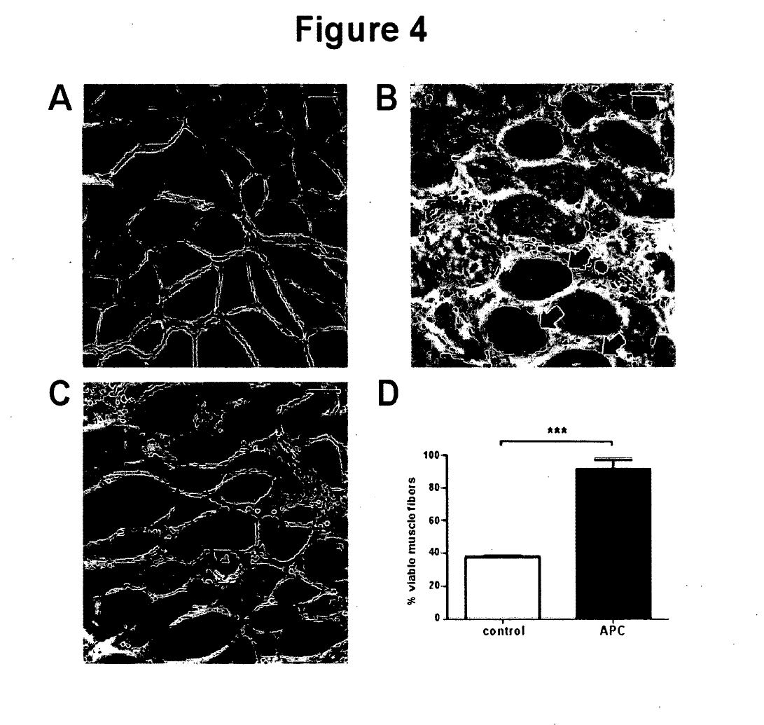

On day 2, quantification of infiltrating polymorphonuclear cells into the flap (FIG. 3) revealed significantly fewer inflammatory cells in the APC group (8.5±4.0 cells per HPF) versus the control group (25.9±4.1 cells per HPF; p<0.05). Moreover, as seen in FIG. 4, a larger percentage of viable muscle fibers were observed within the panniculus camosus layer of the APC treated animals (91.2±5.9 percent) when compared to animals that received PBS (37.5±0.9 percent; p<0.001).

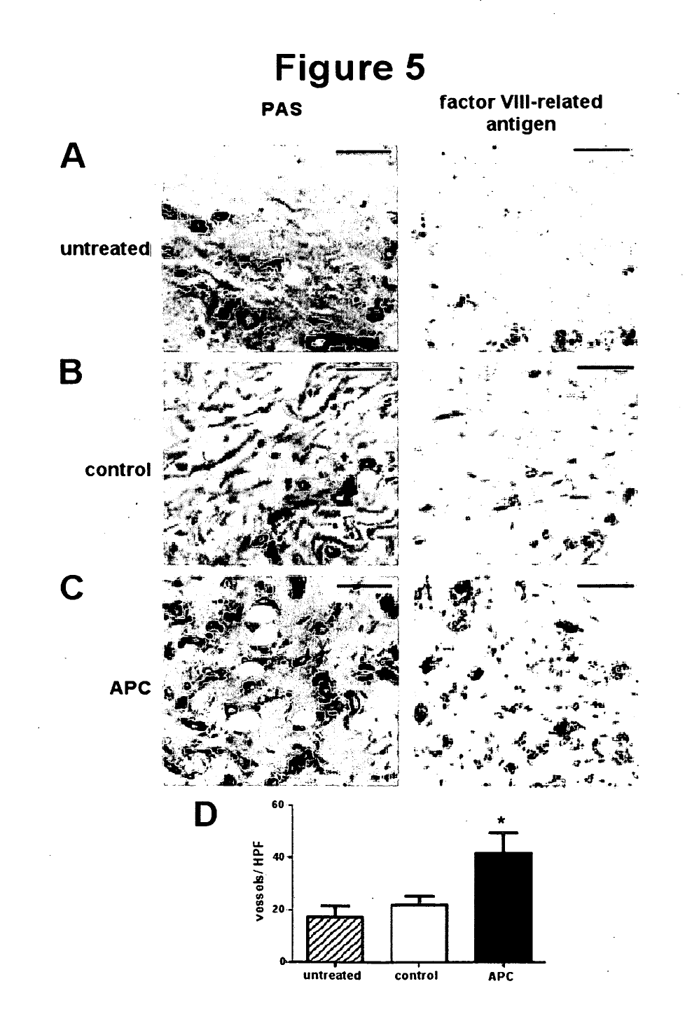

Widespread necrosis characterized by loss of cellularity, particularly within the panniculus camosus, was noted in the control group by day 7 (data not shown). By comparison, the APC group showed maintenance of muscle viability and cellular morphology. Quantification of blood vessels by PAS and factor VIII immunostaining (FIG. 5) revealed an increase in the number of capillaries in the APC group (41.7±7.7 vessels per HPF) in comparison with that in the control group (22.0±3.4 vessels per HPF) or untreated skin (17.2±4.1 vessels per HPF). These findings were significant (n=6; p<0.05).

### Example 5

RNA Extraction and cDNA Synthesis

Full-thickness 3 mm punch biopsies were performed at 3 or 24 hours following flap elevation (n=4, per group, per time-point). These two time-points have consistently been shown to be those at which major transcriptional changes occur during flap ischemia (Chen et al., J Surg Res; 140:45 (2007); Zhang et al., J Reconstr Microsurg; 22:641 (2006); Michlits et al., Wound Repair Regen; 15:360 (2007); Huang et al., Circ J; 70:1070 (2006)). Biopsies were taken from the midline of the flap, 2 cm proximal to its caudal end. This site was chosen as it lies within choke vessel territory between the deep circumflex iliac, lateral thoracic and posterior intercostal arteries (Yang et al., J Surg Res; 87:164 (1999)). Biopsies were placed in ice-cold TRIzol reagent (Invitrogen, Carlsbad, Calif.) and homogenized with a tissue grinder (Pellet Pestle; Kimble Kontes, Vineland, N.J.). Total RNA was isolated by chloroform extraction and isopropanol precipitation, washed in ethanol, resuspended in 20 μl of diethyl pyrocarbonate water, and treated with 3 IU of RNase-free DNase (Promega, Madison, Wis.) at 37° C. for 40 minutes. Spectrophotometric RNA quantification was performed (NanoDrop ND-100; NanoDrop Technologies, Wilmington, Del.) and purity assessed by A_{260 nm}/A_{280 nm} ratio (acceptable ratio was >1.7). SuperScript First-Strand Synthesis System (Invitrogen) was used to reverse transcribe mRNA into cDNA. For each sample, 2 μl oligo(dT), 2 μl dNTP mix, and 5 μg total RNA were diluted with diethyl pyrocarbonate water to 26 μl. The tube was heated at 65° C. for 5 minutes. A reaction mixture containing 8 μl first strand buffer, 2 μl 0.1M DTT, 2 μl RNasin and 2 μl Superscript III reverse transcriptase was added to each tube. The transcription reaction was run at 50° C. for 50 minutes and heat-inactivated at 70° C. for 15 minutes.

### Example 6

### Real-Time Polymerase Chain Reaction

The genes of interest, their primer sequences and melting temperatures are presented in Table 1.

TABLE 1

Custom Primer Sequences for Rat Gene Transcripts
Analyzed in these Experiments

| | | |
|---|---|---|
| Gene transcript | Forward and Reverse Primers | Melting Temperature |

| | | |
|---|---|---|
| ICAM-1 | TCCAATTCACACTGAATGCCAGCC | 60.0° C. |
| | AAGCAGTCCTTCTTGTCCAGGTGA | 60.1° C. |
| TNF-α | CTGGCCAATGGCATGGATCTCAAA | 60.0° C. |
| | ATGAAATGGCAAATCGGCTGACGG | 60.4° C. |
| IL-1β | ACCTGCTAGTGTGTGATGTTCCCA | 60.1° C. |
| | AGGTGGAGAGCTTTCAGCTCACAT | 60.2° C. |
| Egr-1 | TCTGAATAACGAGAAGGCGCTGGT | 60.2° C. |
| | ACAAGGCCACTGACTAGGCTGAAA | 60.4° C. |
| VEGF | TCCAATTGAGACCCTGGTGGACAT | 60.1° C. |
| | TCTCCTATGTGCTGGCTTTGGTGA | 60.2° C. |
| VEGFR2 | AGTGGCTAAGGGCATGGAGTTCTT | 60.3° C. |
| | GGGCCAAGCCAAAGTCACAGATTT | 60.2° C. |
| Bax | TTGCTGATGGCAACTTCAACTGGG | 60.2° C. |
| | TGTCCAGCCCATGATGGTTCTGAT | 60.4° C. |
| Bc1-2 | TTGTGGCCTTCTTTGAGTTCGGTG | 59.8° C. |
| | TCATCCACAGAGCGATGTTGTCCA | 60.3° C. |
| GAPDH | TGATGCTGGTGCTGAGTATGTCGT | 60.3° C. |
| | TCTCGTGGTTCACACCCATCACAA | 60.4° C. |

Abbreviations in Table 1 have the following meanings: TNF, tumor necrosis factor; IL, interleukin; ICAM, intercellular adhesion molecule; Bax, Bc1-2 associated X protein; Bc1, B-cell lymphoma protein; Egr, early growth response factor; VEGF, vascular endothelial growth factor; VEGFR, vascular endothelial growth receptor; GAPDH, gluteraldehyde-3-phosphate dehydrogenase.

These specific genes were selected on the basis of their known or putative role in APC's mechanism of action. Briefly, genomic and mRNA sequences for the genes of interest were obtained through the National Centre for Biotechnology Information's GenBank (National Institutes of Health, Bethesda, Md.). High-stringency reverse transcription polymerase chain reaction primers were designed using Integrated DNA Technologies' PrimerQuest tool (Coralville, Iowa). Primer sequences were checked using the RATMAP genome database (Goteborg University, Sweden) to ensure that forward and/or reverse primers in each pair bridged an exon-exon junction.

Real-time PCR was performed using the Rotor-Gene device (RG-3000; Corbett Research, Sydney, Australia). Reactions (total volume 25 μL) used 5 ng cDNA, 8 μl SYBR® Green I master mix (Invitrogen) and 1 μM of each gene-specific primer. The cycling conditions were: 95° C. for 4 minutes; 45 cycles of 95° C. for 20 seconds, 58° C. for 20 seconds and 72° C. for 20 seconds; and a final elongation step at 72° C. for 4 minutes. PCR product specificity was confirmed by dissociation curve. Each run included a nontemplate control. Fluorescence was acquired at 74° C. Results were evaluated with the Rotor-Gene Analysis Software 6.0 using the 2^{ΔΔCt} relative quantification technique (Pfaffl, M. W., Real-time PCR; 1st Ed. (2006)). Gluteraldehyde-3-phosphate dehydrogenase (GAPDH) served as reference housekeeping gene and samples taken from untreated rat skin served as baseline calibrators. All reactions were repeated in duplicate.

### Example 7

### APC Modulates Expression of Pro-Inflammatory and Pro-Angiogenic Genes

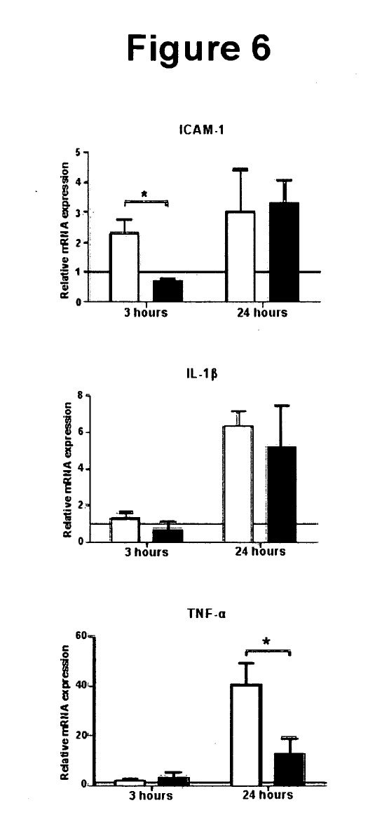

Real-time PCR analysis of postoperative animal flap tissue revealed reduced levels of several pro-inflammatory gene transcripts (FIG. 6). ICAM-1, an adhesion molecule pivotal to inflammatory cell trafficking, was noted to be reduced by two-thirds in APC animals as compared to control animals at 3 hours (p<0.05). No difference was observed between APC and control groups with regards to transcript levels of the pro-inflammatory cytokine IL-1β at either of the time-points tested. On the other hand, a marked decrease in TNF-α transcripts was noted at 24 hours in the APC group as compared to the control group (p<0.05).

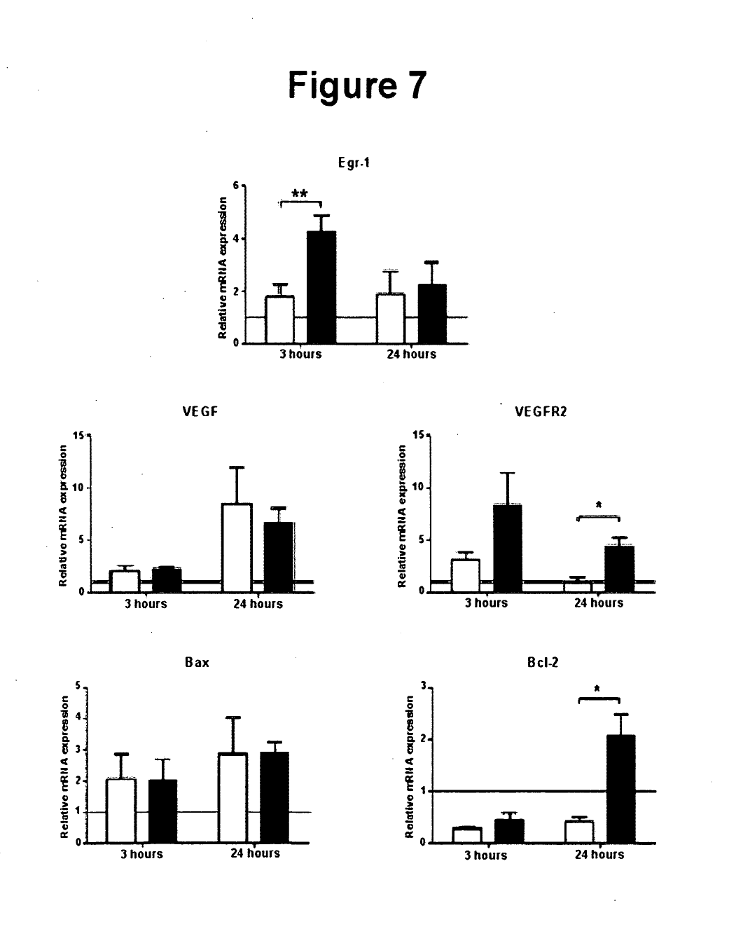

APC led to the upregulation of a number of pro-angiogenic gene transcripts (FIG. 7). Transcripts for early growth response factor 1 (Egr-1) were noted to be two-fold higher at 3 hours following flap elevation in APC treated animals as compared to control animals (p<0.01). No difference was noted between the APC and control groups at 24 hours. Although transcripts for VEGF were observed to be increased from baseline at 24 hours, no difference was noted between APC and control groups. Interestingly, a marked difference in the factor's receptor, VEGFR2, was noted between APC and control groups at 24 hours, with APC treatment producing a greater than four-fold increase in VEGFR2 transcription (p<0.05). No differences were noted between APC and control groups with regards to pro-apoptotic Bax transcription levels. In contrast, levels of the anti-apoptotic mediator Bcl-2 were reduced below baseline at 3 hours in both APC and control groups but were elevated in the APC group at 24 hours (p<0.05).

### Example 8

### Early Preoperative APC Treatment Leads to Near-Complete Flap Survival

Given the rapid effect of APC on gene transcript levels observed in the postoperative treatment group, it was hypothesized that APC treatment initiated prior to surgery would lead to increased cytoprotective gene expression at the time of surgery, leading to further improvements in flap survival.

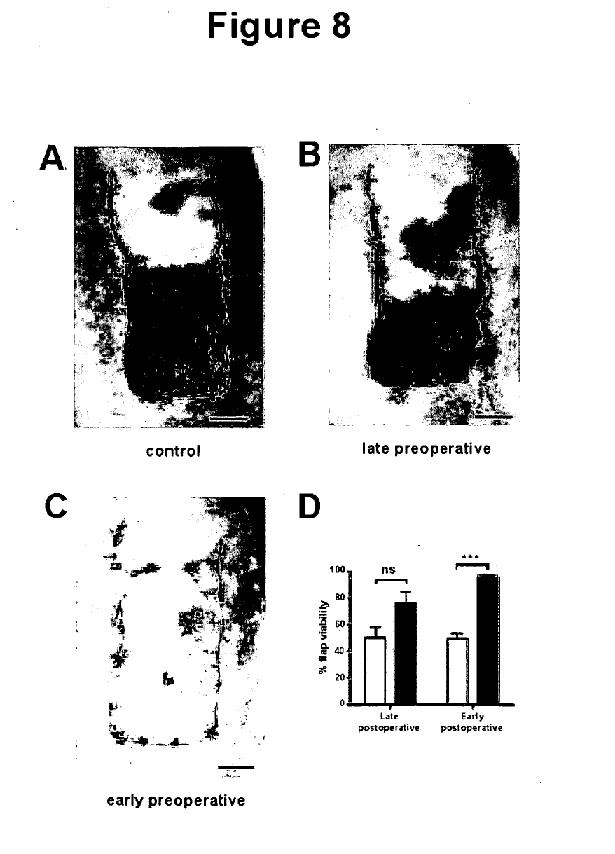

As seen in FIG. 8, APC treatment initiated 45 minutes prior to surgery (late preoperative group) led to improved flap survival compared to control animals (75.9±9.1 percent for APC group versus 50.9±7.1 percent for control group), however, this was not significant (p=0.068). In addition, and more concerning, the late preoperative group displayed a dramatic increase in bleeding during flap elevation with diffuse hemorrhage into the substance of the flap.

In an attempt to circumvent the risk of hemorrhage, APC treatment was initiated three hours before flap elevation, at which point APC's anticoagulant activity, with a half-life of approximately 20 minutes, would be negligible (Hoffmann et al., Crit Care Med; 32:1011 (2004)). In this early preoperative group, the benefit of APC on flap survival was even more marked, with near-complete flap survival in those animals that received APC (96.1±1.1 percent) as compared to those animals that received PBS (50.1±3.3 percent). This difference was highly significant (p<0.001). No increased bleeding was noted intraoperatively. Real-time PCR analysis at 24 hours in the early preoperative group revealed similar changes to those seen in the postoperative group, with APC administration resulting in a significant downregulation of TNF-α transcription (1.6±0.3 for APC group versus 21.4±1.7 for control group, untreated skin level=1, p<0.01), and upregulation of VEGFR2 (8.7±1.5 for APC group versus 2.0±0.5 for control group, untreated skin level=1, p<0.05) and Bcl-2 (2.5±0.7 for APC group versus 0.5±0.2 for control group, untreated skin level=1, p<0.05) transcripts following surgery. Histological examination revealed maintenance of tissue architecture and morphology (data not shown). There were no statistical differences in flap survival between control animals from the postoperative, late preoperative and early preoperative groups.

To determine if the number of APC treatments could be reduced without compromising the observed flap viability improvement seen in the early preoperative group, five additional rats were treated as described for this group, with the exception that the third APC injection at 24 hours was withheld. Although the difference in flap viability relative to control animals observed was significant (70.0±7.2 percent vs. 50.2±3.2 percent, p<0.05), the percentage flap viability was not as great as that seen in the early preoperative group receiving three APC injections.

### Example 9

### Statistical Analysis

All values were expressed as the mean±SEM. For flap viability rate comparisons, a two-tailed t test assuming equal variances was used. The non-parametric Mann-Whitney test was used for statistical analysis of real-time PCR analysis, and one-way ANOVA applied for histological data. Statistical significance was set at p<0.05.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

1. A method for treating or preventing ischemic injury in a tissue flap in a subject, the method comprising administering to the subject a therapeutically effective amount of an agent comprising one or more of; (i) an activated protein C (APC), (ii) a functional fragment of an APC, (iii) an APC mimetic compound, and (iv) a derivative of APC, optionally in admixture with a pharmaceutically-acceptable carrier.

2. The method of claim 1, wherein the agent is APC.

3. The method of claim 2, wherein the agent is human APC.

4. The method of claim 1, wherein the agent is a functional fragment of APC.

5. The method of claim 1, wherein the agent is an APC mimetic compound.

6. The method of claim 1, wherein the agent is a derivative of APC.

7. The method of claim 1, wherein the tissue flap is selected from one or more of a skin flap, fascia flap, and muscle flap.

8. The method of claim 7, wherein two or more tissue flaps are used in combination.

9. The method of claim 1, wherein the tissue flap is a skin flap.

10. The method of claim 1, wherein the tissue flap is a fascia flap.

11. The method of claim 1, wherein the tissue flap is a muscle flap.

12. The method of claim 1, wherein the agent is administered to the subject pre-surgery.

13. The method of claim 1, wherein the agent is administered to the subject pre-surgery and post-surgery.

14. The method of claim 12, wherein the agent is administered to the subject more than one hour pre-surgery.

15. The method of claim 12, wherein the agent is administered to the subject a sufficient amount of time pre-surgery such that the risk of hemorrhage during surgery is minimal.

16. The method of claim 13, wherein the agent is administered to the subject more than one hour post-surgery.

17. The method of claim 13, wherein the agent is administered to the subject a sufficient amount of time post-surgery such that the risk of hemorrhage following surgery is minimal.

18. The method of claim 1, wherein the tissue flap is used for reconstructive surgery.

19. The method of claim 18, wherein the reconstructive surgery is treating a wound or soft tissue defect resulting from cancer ablation.

20. The method of claim 19, wherein the wound or soft tissue defect results from mastectomy, skin cancer excision, or head and neck cancer.

21. The method of claim 18, wherein the reconstructive surgery is treating traumatic injury.

22. The method of claim 1, wherein the agent is administered systemically.

23. The method of claim 22, wherein the agent is administered parenterally.

24. The method of claim 22, wherein the agent is administered intravenously.

25. The method of claim 22, wherein the agent is administered through continuous infusion.

26. The method of claim 22, wherein the agent is administered as a bolus.

27. The method of claim 1, wherein the therapeutically effective amount of the agent is in the range of 0.005 to 2000 μg per kg of body weight.

28. The method of claim 1, wherein the therapeutically effective amount of the agent is in the range of 0.1 to 40 μg per kg of body weight.

29. The method of claim 1, wherein a first and a second surgical procedure are performed on a subject, the second surgical procedure comprising reconstructive surgery using a tissue flap and the first surgical procedure causing the subject to need reconstructive surgery.

30. The method of claim 29, wherein reconstructive surgery using the tissue flap is delayed for a time interval ranging from days to weeks following the first surgical procedure.

31. The method of claim 29, wherein reconstructive surgery using the tissue flap is delayed for a time interval ranging from about 7 to about 21 days following the first surgical procedure.

32. The method of claim 29, wherein reconstructive surgery using the tissue flap is delayed for a time interval of about 7 days following the first surgical procedure.

33. The method of claim 29, wherein reconstructive surgery using the tissue flap is performed contemporaneously with the first surgical procedure.

34. The method of claim 29, wherein the first surgical procedure is treating a wound or soft tissue defect resulting from cancer ablation.

35. The method of claim 34, wherein the wound or soft tissue defect results from mastectomy, skin cancer excision, or head and neck cancer.

36. The method of claim 29, wherein the first surgical procedure is treating traumatic injury.

37. The method of claim 1, wherein the tissue flap is attached using microvascular surgical techniques.

38. The method of claim 1, wherein the tissue flap is autologous relative to a tissue flap recipient.

39. The method of claim 1, wherein the tissue flap is heterologous relative to a tissue flap recipient.

40. A therapeutic composition comprising one or more of: (i) an activated protein C (APC), (ii) a functional fragment of an APC, (iii) an APC mimetic compound, and (iv) a derivative of APC; optionally in admixture with a pharmaceutically acceptable carrier or additive.

41. The composition of claim 40, wherein the composition treats or prevents ischemic injury in a tissue flap in a subject.

42. The composition of claim 40, wherein the therapeutically effective amount of the agent is in the range of 0.005 to 2000 μg per kg of body weight.

43. The composition of claim 40, wherein the therapeutically effective amount of the agent is in the range of 0. 1 to 40 μg per kg of body weight.

44. The composition of claim 40, wherein the tissue flap is used for reconstructive surgery.

45. The composition of claim 43, wherein the reconstructive surgery is treating a wound or soft tissue defect resulting from cancer ablation.

46. The composition of claim 43, wherein the wound or soft tissue defect results from mastectomy, skin cancer excision, or head and neck cancer.

47. A kit comprising one or more of: (i) an activated protein C (APC), (ii) a functional fragment of an APC, (iii) an APC mimetic compound, and (iv) a derivative of APC; optionally in admixture with a pharmaceutically acceptable carrier or additive.

48. The kit of claim 47, wherein the kit is used to treat or prevent ischemic injury in a tissue flap in a subject.

49. The method of claim 13, wherein the agent is administered to the subject more than one hour pre-surgery.

50. The method of claim 13, wherein the agent is administered to the subject a sufficient amount of time pre-surgery such that the risk of hemorrhage during surgery is minimal.
